(19) United States
(12) Patent Application Publication
(10) Pub. No.: US 2011/0177002 A1
(43) Pub. Date: Jul. 21, 2011

(54) CYLIC POLYAMINES FOR BINDING PHOSPHATIDYLSERINE

(75) Inventors: Sabine Zitzmann-Kolbe, Berlin (DE); Ulrike Bauder-Wuest, Schriesheim (DE); Michael Eisenhut, Heidelberg (DE); Doerte Oltmanns, Weinheim (DE)

(73) Assignee: BAYER SCHERING PHARMA AKTIENGESELLSCHAFT, Berlin (DE)

(21) Appl. No.: 13/054,606

(22) PCT Filed: Jul. 14, 2009
(86) PCT No.: PCT/EP2009/005085

§ 371 Date: Apr. 5, 2011

(30) Foreign Application Priority Data
Jul. 18, 2008 (EP) 08160684.0

(51) Int. Cl. A61K 49/06; A61K 49/00; C12N 9/96; A61K 31/395; C07K 14/00; C07K 16/00; C07D 255/02; C07D 257/02; C07D 259/00

(52) U.S. Cl. 424/9.3; 424/9.1; 435/188; 514/183; 530/350; 530/391.1; 540/465

(57) ABSTRACT

The invention pertains to a compound for detecting cell death. According to the invention, the compound comprises a cyclic polyamine unit for the complexation of a bi- or trivalent metal ion. The compound of the invention may contain a reporting tag like fluorescein or a radioisotope including substituent.

The invention pertains to a compound for imaging cell death in vitro and in vivo, as well as to a method for detecting the progression or regression of a tumor in a patient. Furthermore, the invention relates to a pharmaceutical composition, comprising such a compound, a precursor suitable for obtaining it, a method for obtaining it and to a kit for imaging cell death.

Throughout this application, various publications and handbooks are cited. The disclosure of these publications and handbooks are hereby incorporated by reference in its entirety into this application to describe more fully the state of the art to which this invention pertains.

### INTRODUCTION

Therapy control of tumors is one of the main areas of interest during the follow up of cancer patients. This information is currently acquired by a series of conventional investigations including morphological changes, symptomatic responses and clinical chemistry. Radiological techniques like X-ray, computed tomography (CT), ultrasonography (US) and magnetic resonance (MR) are imaging techniques which cover the size and morphology of tumor masses and to some degree alterations of tumor perfusion.

At the same time, nuclear medicine has a series of radiopharmaceuticals at its disposal which are successfully used for the assessment of therapy response. Among those agents, [¹⁸F]FDG represents the main protagonist which is acting as a surrogate marker used to show the influence of e.g. cytostatic drugs on the glucose transport and hexokinase activity in tumors. A decrease in tracer accumulation is expected to indicate treatment response and a better survival. However, interpretation of the signal might be complicated by inflammatory reactions, for example early after radiation therapy. Furthermore, tumor response is shown via a negative signal, i.e. a decrease in tracer uptake. This may be problematic in cases of a moderate accumulation of the tracer prior to therapy. In this respect, tracers providing a positive signal are more desirable.

A few years ago, ^{99m}Tc labeled annexin V was presented as a new agent capable to image cell death in vivo. Apoptosis was visualized in several model systems including myocardial infarction, Fas antibody treated liver and tumors after cytostatic drug application (1, 2). The use of ^{99m}Tc labeled annexin V as an in vivo imaging indicator for apoptosis based on results obtained with the annexin V-fluorescent dye conjugate was used to show apoptotic cells under the microscope (3).

Annexin V has first been isolated from human placenta (4) demonstrating an anticoagulant effect by inhibiting among other factors prothrombinase activity. This effect was attributed to the displacement of coagulation factors from the phospholipid membrane (5).

Annexins are ubiquitous homologous proteins that bind to phospholipids in the presence of calcium. Annexin V, formerly also named as placental anticoagulant protein (PAP) or vascular anticoagulant protein (VAC-α) (5), binds together with Ca²⁺ on externalized phosphatidylserine (PS) present on the cell membranes early after the onset of apoptosis. Under normal conditions, phosphatidylserine resides to about 100% on the interior leaflet of the bi-layered cell membrane where it functions as an anchor for e.g. annexin I, which is involved in transmembrane signaling and internalization processes (6). Due to the activation of effector caspases 3, 6 and 7, which are induced either after intrinsic or extrinsic stimuli, proteolytic inactivation of proteins occurs which maintain under normal conditions the asymmetric distribution of lipids within the cell membrane. Several types of translocase enzymes belong to these proteins, including an ubiquitous Mg'²⁺-ATP dependent aminophospholipid flippase that selectively catalyzes the inward transport of phosphatidylserine and phosphatidylethanolamine, an ATP dependent floppase that moves phospholipids outward with little headgroup specificity, and a Ca²⁺ dependent scramblase that destroys the bilayer asymmetry (7). ATP depletion due to the energy demanding apoptotic process may also hamper the maintenance of the asymmetric lipid distribution.

The high affinity of annexin V for cells which expose phosphatidylserine, exhibiting a K_{d} of <10⁻¹⁰ mol/l (8) is the basis of detecting apoptosis in vivo. The structural background for this strong annexin V-phosphatidylserine interaction is slowly elucidated.

It is known from X-ray structure analyses that this kind of binding functions through the co-complexation of Ca²⁺ with the carboxyl-phospholipid headgroup in collaboration with so called AB, AB′ and DE Ca²⁺-binding sites. These binding sites are located in each of the four tandemly repeated segments of annexin V.

FIG. 1 illustrates the interaction in one of these segments (domain III) with glycerophosphoserine (truncated phosphatidylserine) on Ca²⁺-annexin V binding sites (9). Primary AB and secondary AB′ Ca²⁺ sites are located within the loop between the A and B helices of each domain. They are also called type II and type III Ca²⁺ binding sites, respectively.

It should be noted that the three complexation sites AB, AB′ and DE imply differently coordinated calcium cations with different stabilities, namely AB>AB′>DE. The second complex entity phosphatidylserine coordinates Ca²⁺ in the AB′ site with the carboxyl function of serine and with the negatively charged phosphate oxygen in the AB site. In addition, the α-amino group of serine interacts in this domain with Threonine (Thr) 187.

The interaction of annexin V with phosphatidylserine through jointly used Ca²⁺ is, however, not alone responsible for the strong binding affinity. Multivalent binding must be taken into account, since Ca²⁺ is forming peptide complexes with a stability constant of log K₁ in the range of only 4-5. In combination with phosphatidylserine, this value may increase to some degree because of the bidenticity of phosphatidylserine for Ca²⁺. However, in order to meet the above mentioned small dissociation constant, two or more domains must be involved in the annexin V/Ca²⁺/phosphatidylserine headgroup interactions. These considerations assume that Ca²⁺ is present in a sufficient concentration. The Ca²⁺ dependency was validated by titration in presence of annexin V binding at low membrane occupancy showing strong cooperativity of this interaction with respect to Ca²⁺ (10). Up to 12 Ca²⁺ ions can theoretically be complexed by three binding sites (AB, AB′ and DE) in each of the domains. As many as 10 Ca²⁺ ions have been located in rat annexin V crystal structures all binding at the membrane-facing surface of the protein (9).

Besides the annexin V/phosphatidylserine system, other approaches have been tested for the imaging of cell death resulting from apoptosis but also necrosis. Alternative targets in the apoptotic pathway potentially useful for the in vivo detection of apoptosis are activated caspases (11-13). The two systems tested in this context are based on the binding of irreversible inhibitors or substrates to activated executioner caspases. The hitherto obtained results have been summarized recently (14, 15).

As already mentioned before, annexin V is typically labeled with a fluorescent dye for in vitro assays (3). Although it is utilized extensively in molecular biology, the labeled protein is expensive and moderately unstable. Thus, it is not convenient for high throughput screening assays in drug discovery.

Additionally, approximately 2.5 mmol/l of extracellular Ca²⁺ is needed for complete binding. This can lead to false positive results because most animal cells have a Ca²⁺ dependent scram-blase that can move phosphatidylserine to the cell surface (16). Furthermore, complete annexin binding requires incubation times of up to one hour, which is problematic for kinetic assays (17, 18).

In short, dye labeled annexin V is a useful apoptosis sensor, but it has a number of limitations and there is a need for replacement reagents that are cheap, robust, low-molecular weight, rapidly-binding, membrane-impermeable, and Ca²⁺-independent. Accordingly, the problem underlying the present invention was to provide a molecule that would show improved characteristics as a phosphatidylserine-binding agent and, in particular, to provide a molecule that would show improved characteristics as an apoptosis sensor over annexin V.

In more general term, the aim of the invention is to provide improve molecule enabling the in vivo or in vitro detection of cell death. The improved molecule is a marker specifically binding to dying or dead cells resulting from apoptosis or necrosis.

One of the recent developments introduced zinc bis-(2,2-dipicolylamine) (Zn²⁺ bis-DPA) coordination complex shown in FIG. 2 has been designed to mimic the apoptosis sensing function of annexin V (19, 20). According to FIG. 1, one phosphatidylserine head group is coordinated to two bridging Ca²⁺ ions that are in turn coordinated to one of the four canonical binding sites at the protein surface (9).

### DESCRIPTION OF THE INVENTION

Contrary to the expectation that one of the Zn-coordinating heteroatoms has to be water in order to provide a replacable binding site for phosphatidylserine, the inventors used a compound with at least one cyclic polyamine unit for the complexation of a bi- or trivalent metal ion, in particular cyclen (12[ane]N₄) or cyclam (14[ane]N₄) instead of DPA. Preferably, the cyclen is a cyclic ethylene bridged tetramine, and the cyclam is a mixed ethylene-propylene bridged tetramine, both of which have four nitrogen atoms for Zn²⁺ complexation. The stability constant for this class of compounds is about 10¹⁵ (mol/l)⁻¹ (for cyclam), as compared to 10⁵ (mol/l)⁻¹ for DPA.

Accordingly, in a first aspect of the invention, the underlying problem is solved by a compound or complex that comprises or contains at least one cyclic polyamine unit (C). This cyclic polyamine unit (C) allows for the binding or complexation of a multivalent, in particular of a bi- and/or a trivalent metal ion as a central ion of the complex.

Excluded from the scope of the invention, in one aspect of the invention, is AMD3100 (1,1′-[1,4-Phenylenebis(methylen)]bis [1,4,8,11-tetraazacyclotetradecan]-octohydrobromid-dihy-drate) as such and its derivatives (i.e. substituted AMD3100), which is a compound with cyclic polyamine units. AMD 3100 was designed to treat HIV infections by inhibiting the viral entry through the CD4/CXCR4 receptor assembly. The use of AMD3100 for binding phosphatidylserine and for apoptosis imaging, however, lies within the scope of the invention. 1,1′-[1,3-Phenylenebis-(methylene)]-bis-tris(p-toluenesulphonyl)-1,4,7,10-tetraazacyclododecane is excluded from the invention because it concerns active ingredient active against HIV, compound disclosed below.

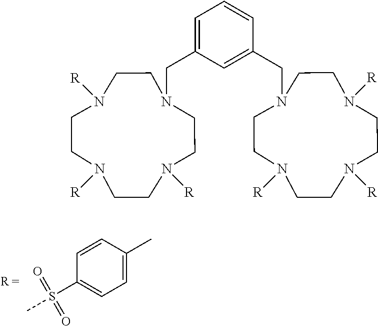

The first aspect is in a first embodiment directed to a compound or complex characterized in that the compound or complex comprises
at least one cyclic polyamine unit (C) for the complexation of bi- or trivalent metal ion or complexing bi- or trivalent metal ion,
and pharmaceutical salt, diastereomere and enantiomere thereof.

Optionally, the compound or complex of the invention comprises further
at least one Tag (T) or linker (L)-Tag (T), and/or
at least one Bridge unit (B) or a bond,

Preferably, the compound or complex comprises
at least one cyclic polyamine unit (C) for the complexation of bi- or trivalent metal ion or complexing bi- or trivalent metal ion,
at least one Tag (T) or linker (L)-Tag (T), and
at least one Bridge unit (B) or a bond.

Preferably, the bi- or trivalent metal ion is a non-radioactive bi- or trivalent metal ion.

Preferably, the compound or complex comprises
at least one cyclic polyamine unit (C) for the complexation of bi- or trivalent metal ion or complexing bi- or trivalent metal ion, and
at least one Tag (T) or linker (L)-Tag (T).

When the multivalent metal ion is a bivalent metal ion, this bivalent metal ion is chosen from the group consisting of Zn²⁺, Ca²⁺, Cu^{2+l, Be}²⁺, Mg²⁺, Sr²⁺, Ba²⁺, Cd²⁺, Cr²⁺, Mn²⁺, Fe²⁺, Co²⁺, Ni²⁺, Pb²⁺, and Hg²⁺.

Preferably, the bivalent metal ion is selected from the group of Zn²⁺, Ca²⁺and Cu²⁺. More preferably, the bivalent metal ion is Zn²⁺.

When the multivalent metal ion is a trivalent metal ion, this trivalent metal ion is chosen from the group consisting of Al³⁺, In³⁺, Cr³⁺, Mn³⁺, Fe³⁺, Co³⁺, Ni³⁺.

Preferably, the trivalent metal ion is selected from the group of Ga³⁺, Mn³⁺, Fe³⁺, Ni³⁺. More preferably, the trivalent metal ion is Ga³⁺.

In embodiments in which the compound or complex of the invention comprises more than one cyclic polyamine unit (C), different metal ions can be complexed by the compound or complex.

In order to facilitate easy detection of the compound or complex, e.g. after administration to a patient or when used in an in vitro detection method for dying cells, the compound or complex further comprises at least one detectable tag (T) (such as a label or signalling group) for detecting the compound or complex, e.g. in the patient. This detectable tag (T) is preferably covalently bond to the compound or complex.

Such a tag (T) can be chosen from the group consisting of fluorescent dyes, enzymes (in particular for the use in an ELISA assay), proteins, magnetic particles, nano particles, liposomes, X-radiation absorbing elements for CT (e.g. iodines, lanthanoids, etc.), elements altering the relaxation times of tissues for MRT (e.g. gadolinium, superparamagnetic Iron Oxide (SPIO)), and radioactive labels.

In particular, the fluorescent dye is selected from the group of a Fluorescein isothiocyanate (FITC), a rhodamine and derivatives, a BODIPY fluorophore (4,4-difluoro-4-bora-3a,4a-diaza-s-indacene, Molecular Probes, Invitrogen), an Alexa Fluor dye (Molecular Probes, Invitrogen), cyanine and merocyanine, Atto (Sigma), Naphtalene derivatives (Dansyl and Prodan derivatives), Pyridyloxazole, Nitrobenzoxadiazole and Benzoxadiazole derivatives, Coumarin derivatives, Pyrene derivatives, Oregon green, eosin, texas red, Cascade blue, Nile red, Cy-3, Cy-5, and derivatives thereof, and any other fluorescent compound which is listed in the Handbook of Fluorescent Probes R. P. Hangland, ISBN 0-9652240-1-5; 1996, pp 8-39. Preferably, the fluorescent dye is Fluorescein isothiocyanate (FITC).

The protein is selected from the group of peroxidase, luziferase, biotin, avidin, an antibody and/or a part of an antibody (e.g. Fc-fragments, diabodies, scFv, F(ab′)2 fragment, Fab . . . ), a toxine, recombinant proteins like scFvs, diabodies, minibodies and suitable proteins known from the skilled person.

The CT label can be chosen from the group consisting of iodines or lanthanoids-containing complexes suitable for computed tomography (CT). The MRT label can be chosen from the group consisting of gadolinium or superparamagnetic Iron Oxide (SPIO)-containing complexes suitable for magnetic resonance computed tomography (MRT) imaging. Suitable CT or MRT elements are well known in the art. The CT/MRT label is a iodine or lanthanoid or gadolinium-contained complex and/or is a moiety or atom that is covalently bound to the compound or complex.

The radioactive label can be chosen from the group consisting of known radioisotopes or their derivatives or radioisotope-contained complexes suitable for positron emission tomography (PET) or single-photon emission computed tomography (SPECT) imaging. Suitable PET radioisotopes (29) are well known in the art (Handbook of Nuclear Chemistry, Vol. 4 (Vol. Ed. F. Rösch; Ed. Vértes, A., Nagy, S., Klencsár, Z.,) Kluver Academic Publishers, 2003; pp 119-202). Suitable radioisotope-contained complexes for SPECT imaging (30) are well known in the art ((Handbook of Nuclear Chemistry, Vol. 4 (Vol. Ed. F. Rösch;Ed. Vértes, A., Nagy, S., Klencsár, Z.,) Kluver Academic Publishers, 2003; pp 279-310). The radioactive label is a radioisotope-contained complex and/or is a moiety or atom that is covalently bond to the compound or complex. The radioisotope is selected from the groups of ^{99m}Tc, ¹⁸F, ¹¹C, ¹²³I, ¹²⁴I, ¹²⁵I, ¹³¹I, ⁶⁴Cu²⁺, ⁶⁷Cu²⁺, ⁸⁹_{Zr}, ⁶⁸_{Ga}³⁺, ⁶⁷_{Ga}³⁺, ¹¹¹In³⁺, ¹⁴C, ³H, ³²P, ⁸⁹Zr and ³³P.

In particular, for positron emission tomography (PET), ¹⁸F, ⁶⁸Ga, ⁶⁴Cu or ¹²⁴ are preferred as positron emitting radioisotopes, more preferably ¹⁸F or ⁶⁸Ga. For single-photon emission computed tomography (SPECT), ¹²³I, ¹²⁵I, ¹¹¹In, and ^{99m}Tc are preferred, more preferably ¹²³I or ^{99m}Tc.

Preferably, the Tag (T) is selected from

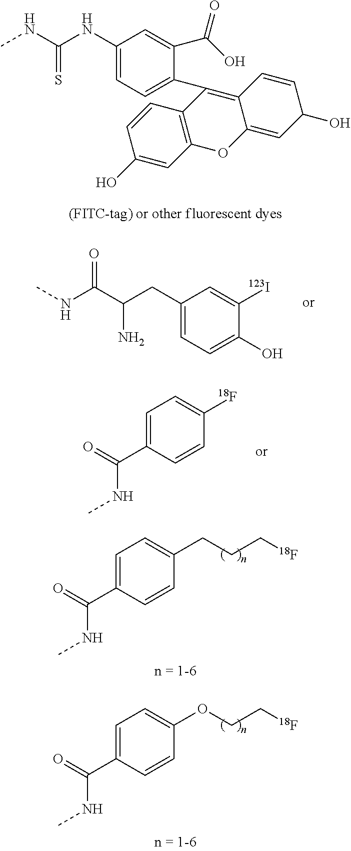

The linker (L) is C₁-C₆ alkyl, O—C₁-C₆ alkyl, C₁-C₆ alkoxy, C₁-C₆ alkylene, (CH₂CH₂O)ₙCH₂CH₂, substituted or non-substituted aryl, substituted or non-substituted O—aryl, substituted or non-substituted heteroaryl, (CH₂)ₙPh, (CH₂CH₂CH₂O)ₙ CH₂CH₂, CO(CH₂)ₙ, [O(CH₂)ₙ—O(CH₂)ₙ]ₘ, or —O(CH₂)ₙ, [O(CH₂)ₙ—O(CH₂)ₙ]ₘ wherein Ph=phenyl and n or m=1, 2, 3, 4, 5, 6.

Preferably, n or m independently from each other is 1 to 4 and more preferably 1 to 3.

Preferably, the linker (L) is C₁-C₆ alkyl, O—C₁-C₆ alkyl, or C₁-C₆ alkoxy.

Preferably, the linker (L) is a PEG chain of formula well known in the art. Preferably, the linker (L) is (CH₂CH₂CH₂O)ₙ CH₂CH₂, CO(CH₂)ₙ, [O(CH₂)ₙ—O(CH₂)ₙ]ₘ, or —O(CH₂)ₙ, [O(CH₂)ₙ—O(CH₂)ₙ]ₘ. More preferably, the linker is O(CH₂)ₙ, [O(CH₂)ₙ—O(CH₂)ₙ]ₘ wherein n is 2 and m is 1.

Besides detectable tags (T), the compound or complex can also be covalently or non-covalently bond to therapeutic compounds, such as drugs, like anti-apoptotic substances which can be administered to a patient, for example after a stroke.

In a preferred embodiment of the invention, the cyclic polyamine unit (C) is a substituted or non-substituted moiety having from 9 to 20 ring members and from 3 to 6 amine nitrogens in the ring spaced by 2 or more carbon atoms from each other. Preferably, the cyclic polyamine unit (C) is
cyclen (an ethylene bridged tetramine) of formula Ia,
cyclam (a mixed ethylene-propylene bridged tetramine) of formula Ib and
cyclen-cyclam mix (a mixed ethylene-propylene bridged tetramine) of formula Ic

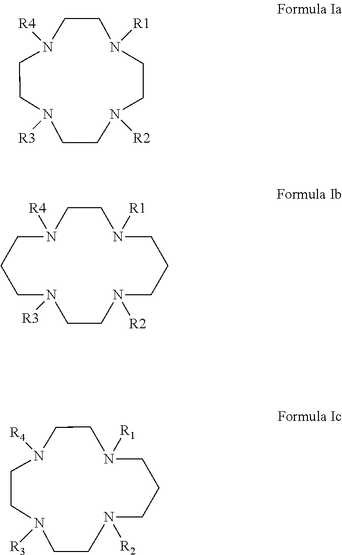

wherein R1, R2, R3 and R4 are independently from each other selected from bond, Hydrogen, C₁-C₆ alkyl, C₁-C₆ alkoxy or Alkyl—COO—, Aryl—COO—.

Preferably, R1, R2, R3 and R4 are independently from each other selected from Hydrogen, C₁-C₄ alkyl, C₁-C₄ alkoxy.

More preferably, the cyclen is a cyclen of formula Ia wherein R1 to R4 is hydrogen.

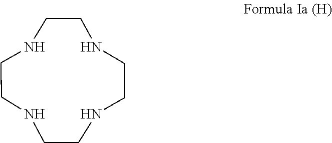

More preferably, the cyclam is a cyclam of formula Ib wherein R1 to R4 is hydrogen.

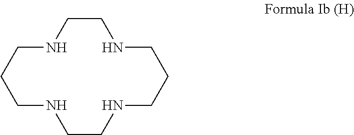

More preferably, the cyclen-cyclam mix is a cyclen-cyclam mix of formula Ic wherein R1 to R4 is hydrogen.

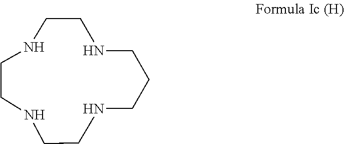

Preferably, the compound or complex comprises 1 to 10 cyclic polyamine units (C). More preferably, the compound or complex comprises 1 to 6 cyclic polyamine units (C). Even more preferably, the compound or complex comprises 1 to 4 cyclic polyamine units (C). Even more preferably, the compound or complex comprises 1 to 2 cyclic polyamine units (C). Even more preferably, the compound or complex comprises 2 cyclic polyamine units (C).

When the invention compound or complex comprises more than 2 cyclic polyamine units (C), then cyclic polyamine units (C) is selected independently from each other from
cyclen (an ethylene bridged tetramine) of formula Ia,
cyclam (a mixed ethylene-propylene bridged tetramine) of formula Ib or
cyclen-cyclam mix (a mixed ethylene-propylene bridged tetramine) of formula Ic.

As described above, a multivalent (in particular a bi- or trivalent) metal ion is complexed by a cyclen, a cyclam or a cyclen-cyclam mix wherein the rings are linked through the ring's N atoms or ring's C atoms.

In a preferred embodiment, the compound or complex contains or comprises at least two cyclic polyamine units that are connected via a bridging unit (B).

In another preferred embodiment, the compound or complex containing or comprising a single cyclic polyamine unit (C) is optionally connected to a bridging unit (B)

As a bridging unit, several structures were surprisingly found and are part of the present invention. Below, preferred bridging units are shown:

In one preferred embodiment of the compound or complex of the invention, the bridging unit (B) is a unit according to formula II wherein the phenyl core structure is substituted or non-substituted at the position not occupied by X1, X2 or X3.

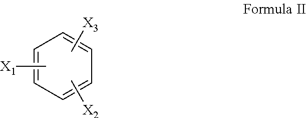

wherein X1, X2 and X3 are independently from each other a H, C₁-C₆ alkyl, C₁-C₆ alkoxy, C₁-C₆ alkylene, C(O), [O(CH₂)ₙ]ₘ, (CH₂CH₂O)ₘ, CH₂OPhCH₂,
(CH₂CH₂CH₂O)ₘ, (CONHPhCH₂), (CONH(CH₂CH₂O)₂CH₂CH₂), [O(CH₂)ₙ]ₘ—NH₂,
or C(O)NH—[CH₂)ₙ—O]ₘ, Ph=phenyl, n=2 to 6 and m=1 to 6.

Optionally, X1, X2 or X3 independently from each other is covalently bond to a cyclic polyamine unit (C), a Tag (T) or linker (L)-Tag (T) with the proviso that X1, X2 or X3 is not H.

Preferably, n=2 to 3 and m=1 to 3.

Preferably, X1, X2 and X3 are independently from each other a C₁-C₆ alkyl, C(O), or (CONH(CH₂CH₂O)₂CH₂CH₂). More preferably, X1, X2 and X3 are independently from each other a methyl or C(O).

Preferably, the bridging unit (B) is a unit according to formula II selected from

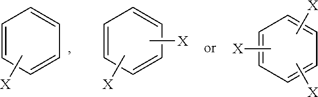

wherein X is X1, X2 or X3.

The bridging unit (B) of formula II is optionally covalently bond to the Tag (T) or linker (L)-Tag (T), see formula IIa1

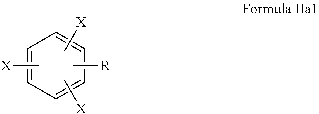

wherein X is X1, X2 or X3 as defined above,
R=Tag (T) or linker (L)-Tag (T) as described above.

Preferably, the bridging unit (B) of formula II is optionally covalently bond to the Tag (T) or linker (L)-Tag (T), see formula IIa2

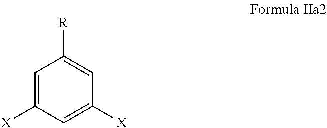

wherein,
wherein X is X1 and/or X2 are defined as above
R=Tag (T) or linker (L)-Tag (T) as described above.

Preferably, the detectable tag (T) is or comprises a fluorescent dye, a radioisotope, an enzyme, a protein, or a labeled substituent as described above.

Preferably, the X moieties of the bridging unit of formula II. IIa1 or IIa2 are identical when there is more than 2 X moieties.

Preferably, the X moieties of the bridging unit of formula II IIa1 or IIa2 differ from each other.

In a second preferred embodiment of the compound or complex of the invention, the bridging unit (B) is a unit according to a substituted or non-substituted formula III which is a substituted benzene-based scaffold.

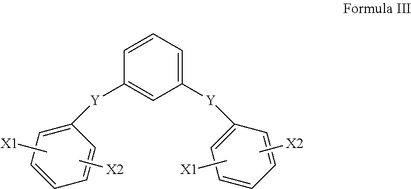

wherein X1 and X2 are independently from each other H, C₁-C₆ alkyl, C₁-C₆ alkoxy, C₁-C₆ alkylene, C(O), [O(CH₂)ₙ]ₘ, (CH₂CH₂O)ₘ, CH₂OPhCH₂, (CH₂CH₂CH₂O)ₘ, (CONHPhCH₂), (CONH(CH₂CH₂O)₂CH₂CH₂), [O(CH₂)ₙ]ₘ—NH₂, or C(O)NH—RCH2)n—[(CH₂)ₙ—O]ₘ, Ph=phenyl, n=2 to 6 and m=1 to 6
Y=(CH₂)ₙ (with n=1, 2, or 3), (CH₂CH₂O)ₘ (with m=0, 1, 2, or 3), O(CH₂)ₚO (with p=2, 3, or 4), or O (independently of each other).

Preferably, X1, and X2 are independently from each other a C₁-C₆ alkyl, C(O), or (CONH(CH₂CH₂O)₂CH₂CH₂). More preferably, X1, and X2 are independently from each other a methyl or C(O) and Y is methyl or C(O).

Optionally, X1 or X2 independently from each other is covalently bond to a cyclic polyamine unit (C), a Tag (T) or linker (L)-Tag (T) with the proviso that X1 or X2 is not H.

The bridging unit (B) of formula III is optionally covalently bond to the Tag (T) or linker (L)-Tag (T) , see formula IIIa1

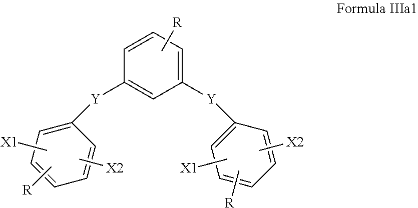

wherein X is X1 and/or X2 are defined as above,
R=H, Tag (T) or linker (L)-Tag (T) as described above.

Preferably, the compound of formula IIIa1 is a compound of formula IIIa1 wherein R is H, Tag (T) or linker (L)-Tag (T) as described above with the proviso that only one R is comprising a Tag (T).

Preferably, the bridging unit (B) of formula III is optionally covalently bond to the Tag (T) or linker (L)-Tag (T) , see formula IIIa2

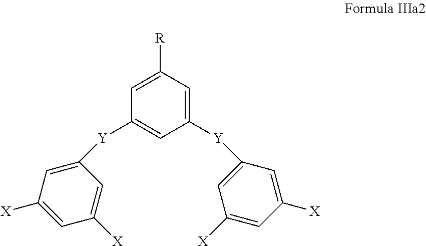

wherein X is X1 and/or X2 are defined as above
R=Tag (T) or linker (L)-Tag (T) as described above.

Preferably, the detectable tag (T) is or comprises a fluorescent dye, a radioisotope, an enzyme, a protein, or a labeled substituent as described above. Preferred embodiments disclosed above are enclosed herein.

In formula III, IIIa1 or IIIa2, the X of the bridging unit (B) is covalently bond to a cyclic polyamine unit (C) through the ring's N atoms or ring's C atoms of the cyclic polyamine unit (C) ring.

Preferably, the X moieties of the bridging unit of formula III, IIIa1 or IIIa2 are identical when there are more than 2 X moieties. Preferably, the X moieties of the bridging unit of formula III, IIIa1 or IIIa2 differ from each other.

In a third preferred embodiment of the compound or complex of the invention, the bridging unit (B) is a unit according to a substituted or non-substituted formula IV which is an adamantane-based scaffold.

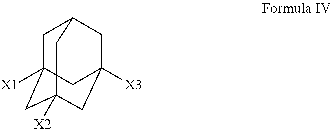

wherein X1, X2 and X3 are defined as above.

X1, X2 or X3 independently from each other is covalently bond to a cyclic polyamine unit (C), a Tag (T) or linker (L)-Tag (T) with the proviso that X1, X2 or X3 is not H.

The bridging unit (B) of formula IV is optionally covalently bond to the Tag (T) or linker (L)-Tag (T), see formula IVa

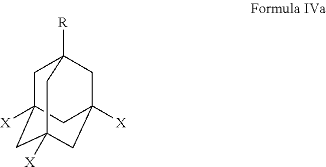

wherein X is X1, X2 and/or X3 and R are defined as above.

Other molecular constructs like dendrimeric structures containing the above described substituents and tags are possible.

The group X as described above is covalently bond to a cyclic polyamine unit, such as cyclen or cyclam either to a C or N atoms of the cyclic polyamine unit, preferably, to one of its N atoms.

In a fourth preferred embodiment of the compound or complex of the invention, the bridging unit (B) is a unit according to a substituted or non-substituted formula V′ and V′a which are peptide-based scaffolds. Formula V′ is based on a peptidic scaffold with α-amino acids, whereas formula V′a is based on a peptidic scaffold with β-amino acids.

Formula V′ is a linear peptide consisting of α-amino acids.

Formula V′a is a linear peptide consisting of β-amino acids.

The bridging unit (B) of formula V′ and V′a are optionally covalently bond to the Tag (T) or linker (L)-Tag (T), see formula V and Va

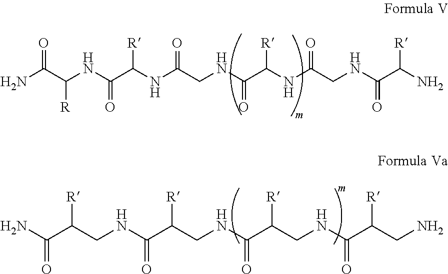

wherein m=1, 2, 3, 4, 5, or 6, and
R′=X
wherein X is X1, X2 and X3 are independently from each other H, C₁-C₆ alkyl, C₁-C₆ alkoxy, C₁-C₆ alkylene, C(O), [O(CH₂)ₙ]ₘ, (CH₂CH₂O)ₘ, CH₂OPhCH₂,
(CH₂CH₂CH₂O)ₘ, (CONHPhCH₂), (CONH(CH₂CH₂O)₂CH₂CH₂), [O(CH₂)ₙ]ₘ—NH₂,
or C(O)NH—[(CH₂)ₙ—O]ₘ, Ph=phenyl, n=2 to 6 and m=1 to 6,
X1, X2 or X3 independently from each other is covalently bond to a cyclic polyamine unit (C), a Tag (T) or linker (L)-Tag (T) with the proviso that X1, X2 or X3 is not H and R=Tag (T) or linker (L)-Tag (T) as described above.

Preferably, X1, X2 and/or X3 independently from each other is an alkyl or aryl bridge connected with a cyclic polyamine unit (C) of formula Ia, formula Ib, or formula Ic. The cyclic polyamine unit (C), such as cyclen or cyclam, is bond either via a C or N, preferably, via one of its N atoms in a compound or complex of formula V or Va.

Furthermore, it is possible to provide functionalized particles like ultrasmall superparamagnetic iron oxide particles (USPIOs) to which the cyclen or cyclam or mixtures thereof of the compound or complex of the invention are bond to predefined surfaces. Methods of binding such functionalized particles to the compound or complex have been described elsewhere (27).

By using the Zn²⁺ complexed and FITC-labeled bis-cyclen derivative shown in FIG. 3 in binding experiments with control and apoptotic Jurkat cells, the inventors surprisingly obtained results comparable to those obtained with fluorescent annexin V. The FACS measurements were reproducible opposing the experiments performed with Zn²⁺ complexed and FITC-labeled bis-DPA complexes.

The positive results acquired with the in vitro apo-test system (15) show that this class of small metal-organic compound or complex is useful for cell death imaging such as apoptosis or necrosis imaging. Table 1 lists preferred compounds or complexes of the invention.

In a further embodiment, the invention is directed to a compound or complex for binding or detecting a phosphatidylserine characterized in that the compound or complex comprises at least one cyclic polyamine unit (C) for the complexation of bi- or trivalent metal ion. Preferably, compound or complex for binding or detecting a phosphatidylserine comprises at least one cyclic polyamine unit (C) for the complexation of non-radioactive bi- or trivalent metal ion or complexing non-radioactive bi- or trivalent metal ion. Preferred disclosed embodiment and preferred alternatives disclosed above are included herein.

In a second aspect, the invention is related to a compound or complex VI characterized in that the compound or complex comprises
at least one cyclic polyamine unit (C) for the complexation of bi- or trivalent metal ion or complexing bi- or trivalent metal ion and
at least one moiety for covalently binding a Tag (T) or linker (L)-Tag (T).

Optionally, the compound or complex VI of the invention comprises further at least one Bridge unit (B) or a bond,

Preferably, the bi- or trivalent metal ion is a non-radioactive bi- or trivalent metal ion.

Preferably, the compound or complex comprises
at least one cyclic polyamine unit (C) for the complexation of bi- or trivalent metal ion or complexing bi- or trivalent metal ion,
at least one moiety for covalently binding Tag (T) or linker (L)-Tag (T), and
at least one Bridge unit (B) or a bond.

The moiety for covalently binding Tag (T) or linker (L)-Tag (T) are known moieties in the art.

The said moiety will enable the covalently binding of a Tag (T) or a linker (L)-Tag (T) to the cyclic polyamine unit (C) or the Bridge unit (B).

Moiety for covalently binding Tag (T) or linker (L)-Tag (T) wherein the Tag (T) is a fluorescent dye, like for example, a Fluorescein isothiocyanate (FITC), a rhodamine, a BODIPY fluorophore (4,4-difluoro-4-bora-3a,4a-diaza-s-indacene, Molecular Probes), an Alexa Fluor dye (Molecular Probes), Cy-3, Cy-5, and derivatives thereof, or any other fluorescent compound which is for example listed in the Handbook of Fluorescent Probes R. P. Hangland, ISBN 0-9652240-1-5; 1996, pp 8-39)

Moiety for covalently binding Tag (T) or linker (L)-Tag (T) wherein the Tag (T) is a protein, is well known in the art. Handbooks provide method and reagents suitable for binding a Tag when the Tag is a protein.

Moiety for covalently binding Tag (T) or linker (L)-Tag (T) wherein the Tag (T) is a radioactive label, is selected from the group of suitable PET radioisotopes are well known in the art (Handbook of Nuclear Chemistry, Vol. 4 (Vol. Ed. F. Rösch;Ed. Vértes, A., Nagy, S., Klencsár, Z.) Kluver Academic Publishers, 2003; pp 119-202). Suitable radioisotope containing complexes for SPECT imaging are well known in the art (ibid.; pp 279-310). The embodiments disclosed above apply here.

In a third aspect, the invention is related to a compound or complex as described herein used for binding and detecting dying cells, both in vitro and in vivo. In particular, such use makes apoptosis or necrosis imaging in vivo possible, by using positron emission tomography (PET), single-photon emission computed tomography (SPECT), magnetic resonance imaging (MRI), and/or optical imaging, preferably positron emission tomography (PET).

In one embodiment, the compound or complex characterized in that the compound or complex comprises
at least one cyclic polyamine unit (C) for the complexation of non-radioactive bi- or trivalent metal ion or complexing non-radioactive bi- or trivalent metal ion.

In one embodiment, the compound or complex characterized in that the compound or complex comprises
at least one cyclic polyamine unit (C) for the complexation of non-radioactive bi- or trivalent metal ion or complexing non-radioactive bi- or trivalent metal ion for the manufacture of pharmaceutical composition or the use as a imaging agent for detecting dying cells.

Cell death occurs through either necrosis or apoptosis. Necrosis (31) is due to unexpected and accidental cell damage and is an unregulated process in which the cells do not usually send the same chemical signals to the immune system that cells undergoing apoptosis do. This prevents nearby phagocytes from locating and engulfing the dead cells, leading to a build up of dead tissue and cell debris at or near the site of the cell death. A number of toxic chemical and physical events can cause necrosis like toxins, radiation, heat, trauma, lack of oxygen. Apoptosis (31) is a regulated process of self-destruction of a cell, triggered by non-reparable cell damage, such as DNA-damage, mutations in essential proteins or developmental processes. Programmed cell death involves a series of biochemical events leading to cell death and leading to processes of disposal of cellular debris whose results do not damage the organism.

In a further embodiment, the invention relates to the use of invention cyclic polyamine compound or complex as described herein or composition thereof for in vivo or in vitro detection of cell death resulting from apoptosis or necrosis. More preferably, the use is for in vitro detection. In vitro detection can be conducted by FACS, microspectrometry, microscopy or ELISA. More preferably, the use is for in vivo detection. In vivo detection can be conducted by PET, SPECT, CT or MRT.

Preferably, the invention relates to the use of invention cyclic polyamine compounds or complexes as described herein or composition thereof for in vivo or in vitro detection apoptosis or necrosis.

Advantageously, such a method allows for the assessment of a patient suffering from a tumor, from an infection, and/or from an inflammation, from injury, from stroke, from heart attack, to an anti-tumor therapy, to anti-infection therapy and/or to anti-inflammatory therapy, respectively as the measurement of cell death which is directly correlated to disease progression or regression and also allows monitoring of therapy efficacy. In particular, the compound or complex of the invention is advantageous as the decrease in its uptake is readily detectable.

Preferably the use is for the manufacture of pharmaceutical composition or use as a imaging agent for detecting necrosis.

Preferably the use is for the manufacture of pharmaceutical composition or use as a imaging agent for detecting apoptosis.

Accordingly, a method for in vivo or in vitro detection of cell death that occurs during apoptosis or necrosis, and thereby the progression or regression of a tumor, of an infection, and/or of an inflammation in a tissue of a patient is also provided. Such a method comprises or contains the following steps:
a) administering a compound or complex as described herein to a patient, and
b) measuring the amount and/or the concentration of the compound or complex in the tissue.

Accordingly, a method for detecting phosphatidylserine and thereby the progression or regression of a tumor, of an infection, and/or of an inflammation, stroke, heart attack, injury in a tissue of a patient is also provided. Such a method comprises or contains the following steps:
a) administering a compound or complex as described herein to a patient, and
b) measuring the amount and/or the concentration of the compound or complex in the tissue.

The patient can be any mammal and is preferably a human being. The measurement of the amount and/or the concentration of the compound or complex in the tissue of interest, i.e. the diseased tissue, can be performed using PET, SPECT, MRI, CT and/or optical imaging.

A compound or complex used in this detection method preferably comprises at least two cyclic polyamine units that are connected via a bridging unit for connecting at least two cyclic polyamine units with each other. The bridging unit is preferably selected from the group consisting of the compound or complex of formulas II to V as shown above.

The invention concerns the use of AMD3100 (1,1′-[1,4-Phenylenebis(methylen)]bis [1,4,8,11-tetraazacyclotetradecan]-octohydrobromid-dihy-drate) for binding and detecting dying or dead cells or for binding and detecting phosphatidylserine.

Preferably, the above use is for binding and detecting dying and dead cells wherein dying or dead cells resulting from apoptosis or necrosis.

The embodiments disclosed above apply here.

The compounds of the invention may be used for the detection and diagnosis of a wide variety of medical conditions, characterized by dying cells.

Examples of clinical conditions characterized by dying cells are as follows:
Diseases which are characterized by occurrence of excessive apoptosis, such as degenerative disorders, neurodegenerative disorders (e.g., Parkinson's disease, Alzheimer's disease, Huntington chorea), AIDS, Motor Neuron Diseases such as amyotrophic lateral sclerosis (ALS), Prion Diseases (e.g. Creutzfeldt-Jacob disease), myelodysplastic syndromes, ischemic or toxic insults, graft cell loss during transplant rejection; tumors, and especially highly malignant/aggressive tumors, are also often characterized by increased apoptosis in addition to excessive tissue proliferation.
Vascular disorders, such as myocardial infarction, cerebral stroke, deep vein thrombosis, disseminated intravascular coagulation (DIC), thrombotic thrombocytopenic purpura (TIP), sickle cell diseases, thalassemia, antiphospholipid antibody syndrome, systemic lupus erythematosus.
Inflammatory disorders, and/or diseases associated with immune-mediated etiology or pathogenesis; auto-immune disorders such as antiphospholipid antibody syndrome, systemic lupus erythematosus, connective tissue disorders, such as rheumatoid arthritis, scleroderma; thyroiditis; dermatological disorders such as pemphigus or erythema nodosum; autoimmune hematological disorders; autoimmune neurological disorders such as myasthenia gravis, multiple sclerosis, inflammatory bowel disorders, such as ulcerative colitis and vasculitis.
Atherosclerotic plaques, and especially plaques that are unstable, vulnerable and prone to rupture, are also characterized by dying cells, such as apoptotic macrophages, apoptotic smooth muscle cells or apoptotic endothelial cells.

The methods of the present invention may be used to detect and/or diagnose one or more of the aforementioned clinical conditions in a human patient or animal subject. Additionally, the detection may also be carried out in a person already known to have the respective disease, for the purpose of evaluating the disease severity or in order to monitor

In a fourth aspect, the invention is related to a method for synthesizing the invention compound or complex as described herein.

In one embodiment the invention is related to a method for synthesizing compound or complex comprising at least one cyclic polyamine unit (C) for the complexation of non-radioactive bi- or trivalent metal ion or complexing non-radioactive bi- or trivalent metal ion. In a further embodiment the invention is related to a method comprising the step of reacting a compound or complex of formula VI comprising
at least one cyclic polyamine unit (C) for the complexation of non-radioactive bi- or trivalent metal ion or complexing non-radioactive bi- or trivalent metal ion and
at least one moiety for covalently binding a Tag (T) or linker (L)-Tag (T) for obtaining a compound or complex comprising
at least one cyclic polyamine unit (C) for the complexation of non-radioactive bi- or trivalent metal ion or complexing non-radioactive bi- or trivalent metal ion, and
at least one Tag (T) or linker (L)-Tag (T).

According to this method, at least two cyclic polyamine units are connected via a bridging unit for connecting at least two cyclic polyamine units with each other. Preferably, the bridging unit is selected from the group consisting of the compound or complex of formulas II to V.

The embodiments disclosed above apply here.

In a fifth aspect, the invention is related to a pharmaceutical composition comprising a compound or complex as described herein, and possibly further a suitable carrier and/or additive wherein the compound or complex comprises at least one cyclic polyamine unit (C) for the complexation of non-radioactive bi- or trivalent metal ion or complexing non-radioactive bi- or trivalent metal ion.

The embodiments disclosed above apply here.
Pharmaceutical compositions for therapeutics, as well as diagnostic compositions of the invention may be administered by any of the known routes, inter alia, oral, intravenous, intraperitoneal, intramuscular, subcutaneous, sublingual, intraocular, intranasal or topical administration, or intracerebral administration. The carrier should be selected in accordance with the desired mode of administration, and include any known components, e.g. solvents; emulgators, excipients, talc; flavors; colors, etc. The pharmaceutical composition may comprise, if desired, other pharmaceutically-active compounds which are used to treat the disease, eliminate side effects or augment the activity of the active component.

In a sixth aspect, the invention is related to a kit comprising one of the invention compound or complex. Preferably the kit is useful for binding and detecting phosphatidylserine, particularly in vitro. Such a kit comprises a compound or complex as described herein and appropriate buffers for dissolving and/or diluting the compound or complex, an agent such as an antibody for detecting the compound or complex and/or ELISA plates for performing an ELISA assay.

In a seventh aspect, the invention is related to a method of selectively targeting a medicinally-useful compound to dying cells within a population of cells, the method comprising: contacting the cell population with a compound represented by the structure set forth in any invention compounds as disclosed and claimed in the present application, thereby selectively targeting the medicinally-useful compound to the dying cells within the cell population.

Preferably the compound or complex of the invention are selected from the group below and complexed with bi- or trivalent metal ion thereof

Invention compounds:

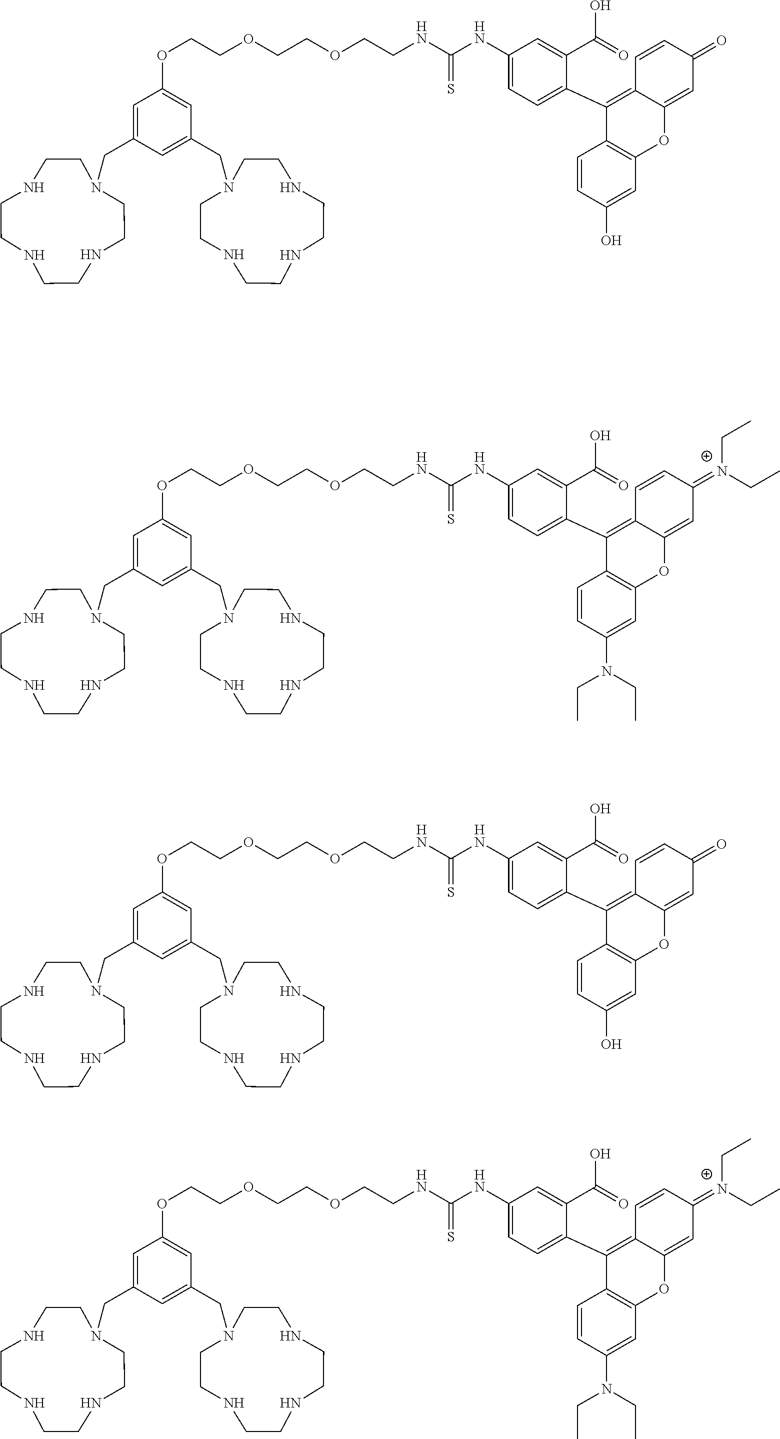

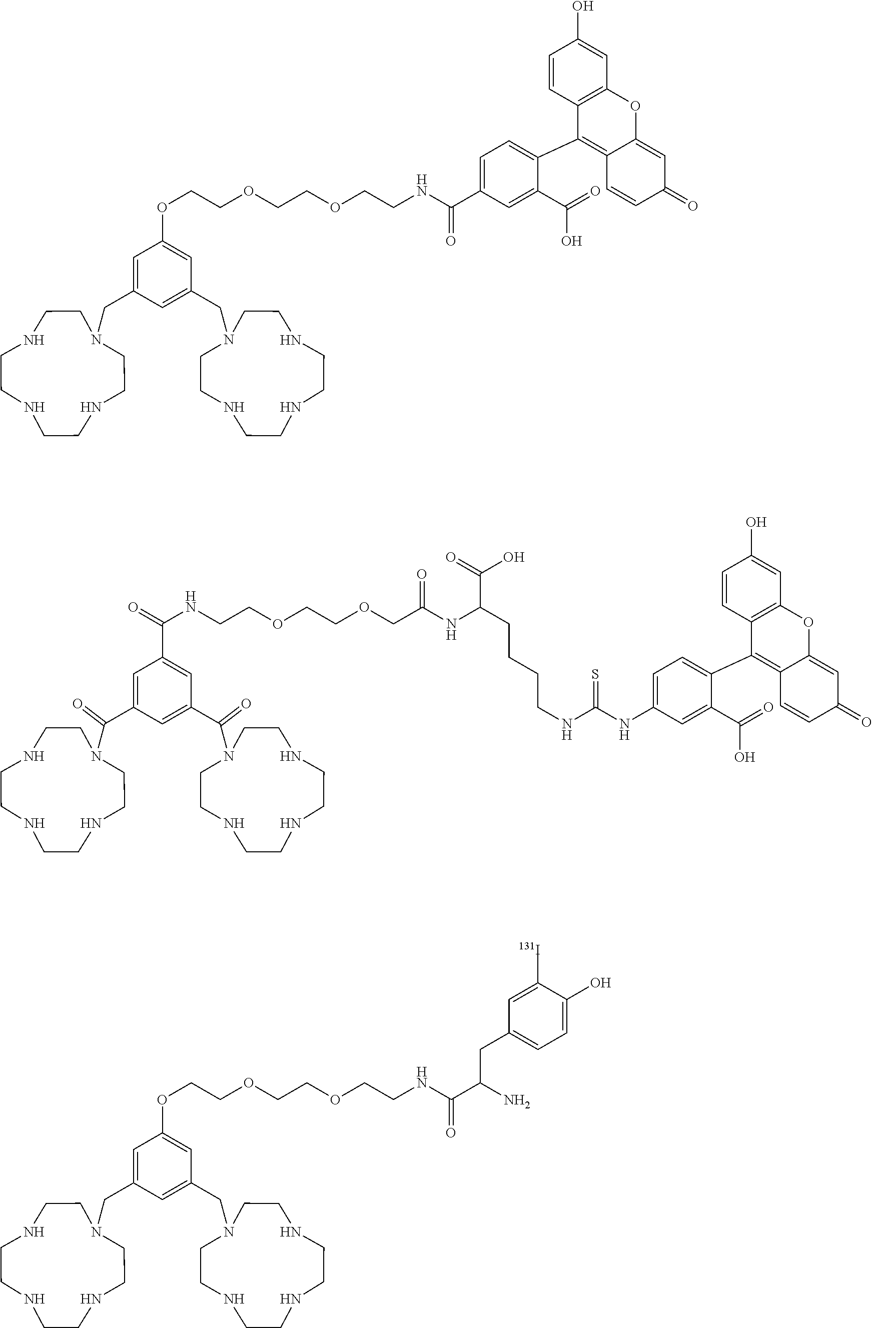

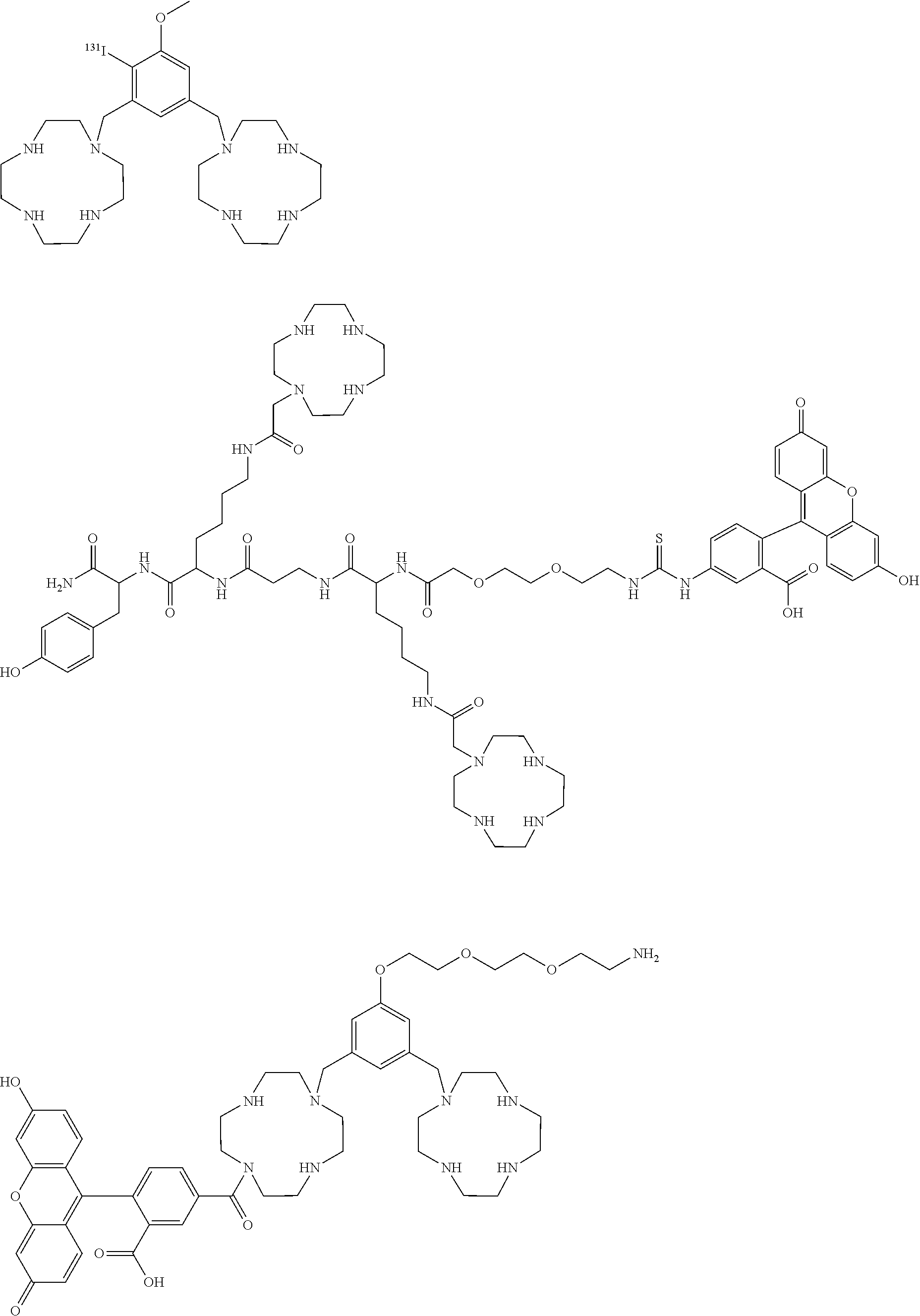

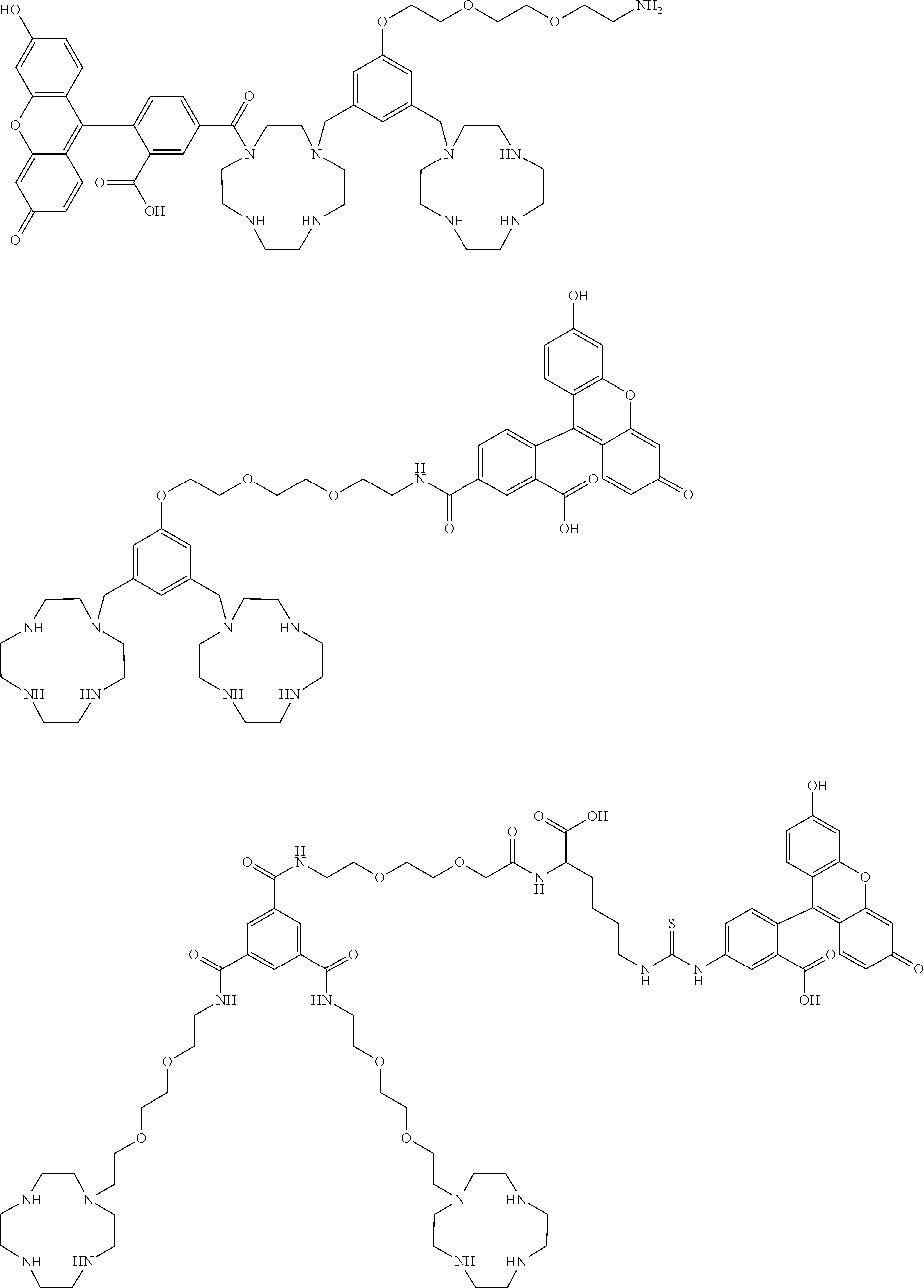

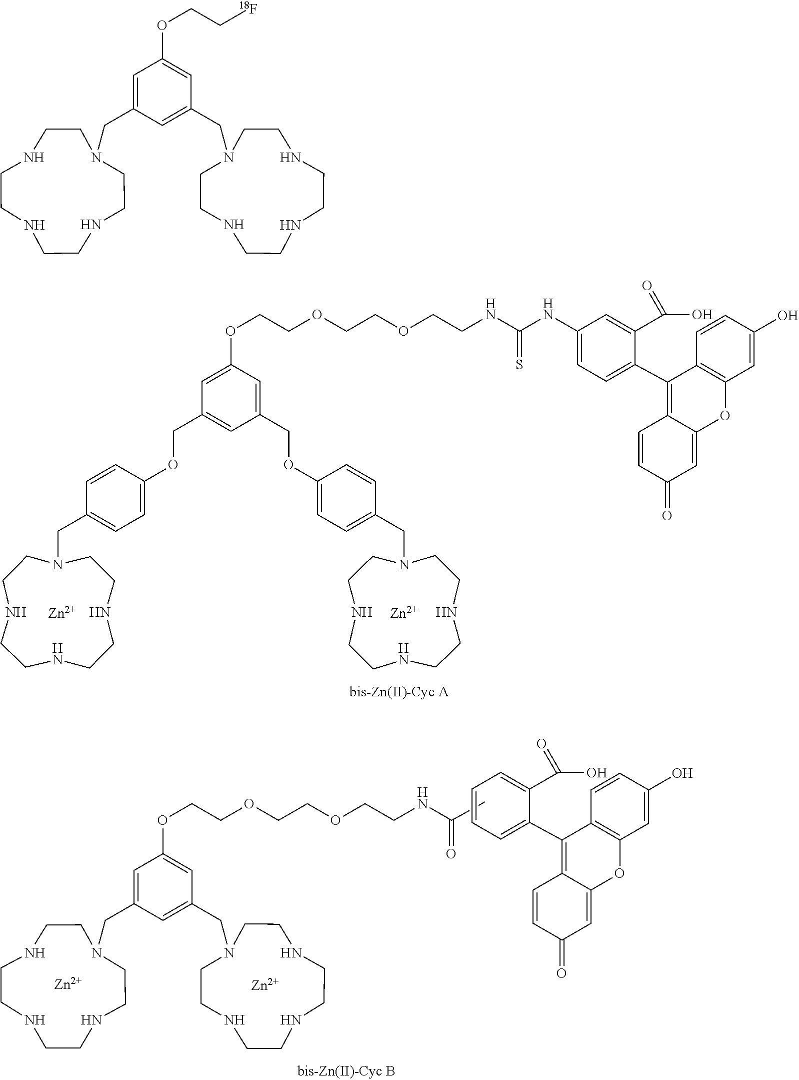

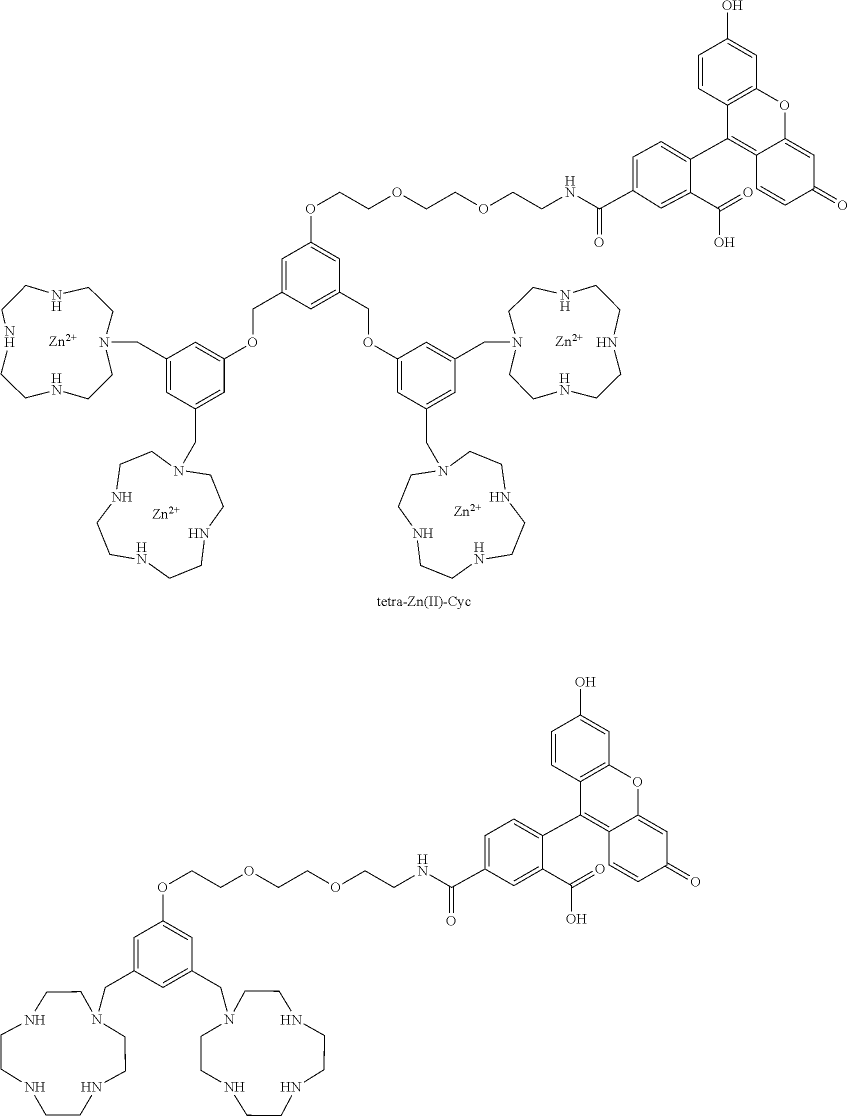

### FIGURES

FIG. 1:

Illustration of the interaction in one of domain III of annexin V with glycerophosphoserine (truncated PS) on Ca²⁺-annexin V binding sites (9). Primary AB and secondary AB′ Ca²⁺ sites are located within the loop between the A and B helices of each domain. They are also called type II and type III Ca²⁺ binding sites, respectively. The structural alignment of glycerophosphoserine (light) in AB and AB′ Ca2⁺-binding sites in third domain of annexin V (shaded) is shown (28).

FIG. 2:

The depiction of a FITC labeled Zn²⁺bis-DPA complex according to the state of the art.

FIG. 3:

The depiction of a preferred compound or complex of the invention, namely a FITC labeled Zn²⁺bis-cyclen complex.

FIG. 4:

Flow cytometry FACS for Annexin V and Propidiumiodide, FL-I, FL-III and FL-I/FL-III-plot.

FIG. 5:

Flow cytometry FACS for Annexin V and Propidiumiodide, FL-I, FL-III and FL-I/FL-III-plot.

FIG. 6:

Biodistribution data of compound 19 for the 3.5 h time point of induction of hepatic apoptosis.

FIG. 7:

PET acquisition of the 18F-Zn complex 19 in transversal section. Radiated tumor (left) and control (right) a) 1 d before the radiation therapy, b) 6 d after radiation, c) 10 d after radiation.

FIG. 8:

Non induced control cells, incubated with compound 1. FL-1 is fluorescence of compound 1, FL-III is fluorescence of PI. Apoptotic cells are found in Q1, necrotic cells in Q2 and Q4, vital cells in Q3. First left graphic is green.

FIG. 9:

Apoptosis induced Jurkat cells, incubated with compound 1. A: FL-1 is fluorescence of compound 1 (green), B: FL-III is fluorescence of PI, (Red). C: Forward and sideward scatter show homogeneous population in region RN1. D: Overlapping fluorescence spectrum was compensated. Apoptotic cells in Q1, necrotic cells in Q2 and Q4, vital cells in Q3.

FIG. 10:

Apoptosis induced Jurkat cells, incubated with compound AB (FL-III) and Annexin-V (FL-I).

FIG. 11:

Apoptosis induced Jurkat cells, incubated with compound 1 (left, green) and compound we (right, green).

FIG. 12:

Apoptosis induced Jurkat cells, incubated with compound 1 complexed with Zn²⁺ (left diagram, green) or compound 1 without Zn²⁺ (right diagram, green).

FIG. 13:

Apoptosis induced Jurkat cells, incubated with compound 1 and PI. The overlay of the green fluorescence from compound 1 (2^{nd} from left) with the red fluorescence from PI (2^{nd} from right) gives the yellow fluorescence seen in the right picture. The left picture shows the transmission picture.

FIG. 14:

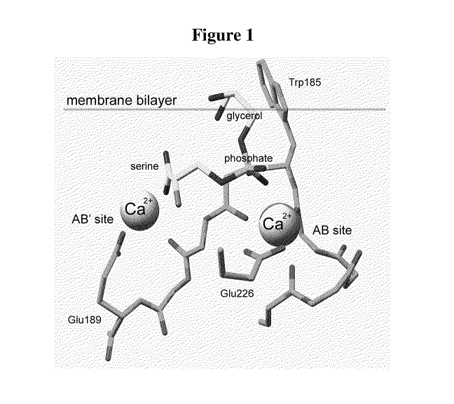

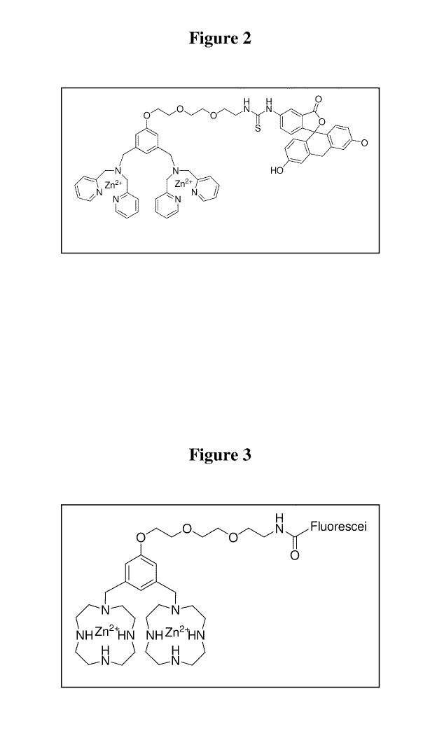

Apoptosis induced Jurkat cells, incubated with compound AB (red) and Annexin-V (green). The overlay of the red fluorescence from compound AB and the green fluorescence from Annexin-V shows labeling of compound AB of both apoptotic and necrotic cells. The left picture shows the transmission picture.

### DEFINITIONS

A cyclic polyamine unit (C) for the complexation of non-radioactive bi- or trivalent metal ion means a cyclic polyamine unit that is able to complex one or more non-radioactive bi- or trivalent metal ion as suitable. Preferably the cyclic polyamine unit is able to complex one non-radioactive bi- or trivalent metal ion.

A cyclic polyamine unit (C) complexing non-radioactive bi- or trivalent metal ion means a cyclic polyamine unit that is complexing or comprising one or more non-radioactive bi- or trivalent metal ion as suitable. Preferably the cyclic polyamine unit is complexing or comprising one non-radioactive bi- or trivalent metal ion.

The term “alkyl” as used herein refers to C₁ to C₆ straight or branched alkyl groups, e. g., methyl, ethyl, propyl, isopropyl, n-butyl, t-butyl, n-pentyl, or neopentyl. Alkyl groups can be perfluorated or substituted by one to five substituents selected from the group consisting of halogen, hydroxyl, C₁-C₄ alkoxy, or C₆-C₁₂ aryl (which can be substituted by one to three halogen atoms). More preferably, alkyl is a C₁ to C₄ or C₁ to C₃ alkyl.

The term “alkenyl” as used herein refers to a straight or branched chain monovalent or divalent radical, containing at least one double bond and having from two to ten carbon atoms, e.g., ethenyl, prop-2-en-1-yl, but-1-enyl, pent-1-enyl, penta-1,4-dienyl, and the like.

The term “alkynyl” as used herein refers to a substituted or unsubstituted straight or branched chain monovalent or divalent radical, containing at least one triple bond and having from two to ten carbon atoms, e.g., ethynyl, prop-1-ynyl, but-1-ynyl, pent-1-ynyl, pent-3-ynyl, and the like.

Alkenyl and alkynyl groups can be substituted by one or more substituents selected from the group consisting of halogen, hydroxyl, alkoxy, —CO₂H, —CO₂Alkyl, —NH₂, —NO₂, —N₃, —CN, C₁-C₂₀ acyl, or C₁-C₆ acyloxy.

The term “aryl” as used herein refers to an aromatic carbocyclic or heterocyclic moiety containing five to 10 ring atoms, e.g., phenyl, naphthyl, furyl, thienyl, pyridyl, pyrazolyl, pyrimidinyl, oxazolyl, pyridazinyl, pyrazinyl, chinolyl, or thiazolyl. Aryl groups can be substituted by one or more substituents selected from the group consisting of halogen, hydroxyl, alkoxy, —CO₂H, —CO₂Alkyl, —NH₂, Alkyl—NH₂, C₁-C₂₀ alkyl-thiolanyl, —NO₂, —N₃, —CN, C₁-C₂₀ alkyl, C₁-C₂₀ acyl, or C₁-C₂₀ acyloxy. The heteroatoms can be oxidized, if this does not cause a loss of aromatic character, e. g., a pyridine moiety can be oxidized to give a pyridine N-oxide.

Whenever the term “substituted” is used, it is meant to indicate that one or more hydrogens on the atom indicated in the expression using “substituted” is replaced with a selection from the indicated group, provided that the indicated atom's normal valency is not exceeded, and that the substitution results in a chemically stable compound, i. e. a compound that is sufficiently robust to survive isolation to a useful degree of purity from a reaction mixture, and formulation into a pharmaceutical composition. The substituent groups may be selected from halogen atoms, hydroxyl groups, nitro, (C₁-C₆)carbonyl, cyano, nitrile, trifluoromethyl, (C₁-C₆)sulfonyl, (C₁-C₆) alkyl, (Cᵢ-C₆)alkoxy and (C₁-C₆)sulfanyl.

Halogen means Chloro, Iodo, Fluoro and bromo. Preferably, halogen means iodo or fluoro.

If a chiral center or another form of an isomeric center is present in a compound according to the present invention, all forms of such stereoisomer, including enantiomers and diastereoisomers, are intended to be covered herein. Compounds containing a chiral center may be used as racemic mixture or as an enantiomerically enriched mixture or the racemic mixture may be separated using well-known techniques and an individual enantiomer maybe used alone. In cases in which compounds have unsaturated carbon-carbon bonds double bonds, both the (Z)-isomer and (E)-isomers are within the scope of this invention. In cases wherein compounds may exist in tautomeric forms, such as keto-enol tautomers, each tautomeric form is contemplated as being included within this invention whether existing in equilibrium or predominantly in one form.

It's well known for a person skilled in the art, that the compounds of the invention, as applicable, are or may be in the form of zwitterions and/or salt at the physiological pH of 7.4.

Preferably the patient is a mammal; most preferably the patient is a human.

While it is possible for the imaging agent to be administered alone, it is preferable to present it as a pharmaceutical formulation comprising at least one imaging agent compound, together with one or more pharmaceutically acceptable carriers, such as diluents or excipients which may include, for example, fillers, extenders, wetting agents, disintegrants, surface-active agents, or lubricants, depending on the nature and mode of administration and the dosage forms. Each carrier must be “acceptable” in the sense of being compatible with the other ingredients of the formulation and not injurious to the patient. The pharmaceutical formulation may optionally include other diagnostic or therapeutic agents. Techniques and formulations may be found, for example, in Remington's Pharmaceutical Sciences. Mack Publishing Co., Easton, Pa. (latest edition).

It should be understood that in addition to the ingredients particularly mentioned above, the formulations of this invention may include other agents conventional in the art having regard to the type of formulation in question, for example, those suitable for oral administration may include such further agents as sweeteners, thickeners and flavouring agents.

Unless otherwise specified, when referring to the compounds of Formula the present invention per se as well as to any pharmaceutical composition thereof the present invention includes all of the hydrates, salts, solvates, complexes, and prodrugs of the compounds of the invention. Prodrugs are any covalently bonded compounds, which releases the active parent pharmaceutical according to the present invention.

The term “prodrug” as used throughout this text means the pharmacologically acceptable derivatives such as esters, amides and phosphates, such that the resulting in vivo biotransformation product of the derivative is the active drug as defined in the compounds of Formula (I). The reference by Goodman and Gilman (The Pharmacological Basis of Therapeutics, 8 ed, McGraw-HiM, Int. Ed. 1992,“Biotransformation of Drugs”, p 13-15) describing prodrugs generally is hereby incorporated. Prodrugs of a compound of the present invention are prepared by modifying functional groups present in the compound in such a way that the modifications are cleaved, either in routine manipulation or in vivo, to the parent compound. Prodrugs of the compounds of the present invention include those compounds wherein for instance a hydroxyl group, such as the hydroxyl group on the asymmetric carbon atom, or an amino group is bonded to any group that, when the prodrug is administered to a patient, cleaves to form a free hydroxyl or free amino, respectively.

Cell death occurs through either necrosis or apoptosis.

Necrosis is due to unexpected and accidental cell damage and is an unregulated process in which the cells do not usually send the same chemical signals to the immune system that cells undergoing apoptosis do. This prevents nearby phagocytes from locating and engulfing the dead cells, leading to a build up of dead tissue and cell debris at or near the site of the cell death. A number of toxic chemical and physical events can cause necrosis like toxins, radiation, heat, trauma, lack of oxygen.

Apoptosis is a regulated process of self-destruction of a cell, triggered by non-reparable cell damage, such as DNA-damage, mutations in essential proteins or developmental processes Programmed cell death involves a series of biochemical events leading to cell death and leading to processes of disposal of cellular debris whose results do not damage the organism.
The term “dying cell” refers herein to a cell that undergoes a death process. Such cells may be exemplified by cells undergoing an apoptotic process, or neuronal cells undergoing neurodegeneration.

The following disclosure is part of the invention. The invention concerns additionally a compound for binding phosphatidylserine,
characterized in that
the compound comprises at least one cyclic polyamine unit for the complexation of a bi- or trivalent metal ion.

2. The compound according to claim 1, wherein the bivalent metal ion is chosen from the group consisting of Zn²⁺, Ca²⁺, Cu²⁺, Be²⁺, Mg²⁺, Sr²⁺, Ba²⁺, Cd²⁺, Cr²⁺, Mn²⁺, Fe²⁺, Co²⁺, Ni²⁺, Pb²⁺, Hg²⁺, and corresponding radiometals of the aforementioned ions.

3. The compound according to claim 1, wherein the trivalent metal ion is chosen from the group consisting of Al³⁺, Ga³⁺, In³⁺, Cr³⁺, Mn³⁺, Fe³⁺, Co³⁺, Ni³⁺, and corresponding radiometals of the aforementioned ions.

4. The compound according to claims 1 to 3, further comprising a tag for detecting the compound.

5. The compound according to claims 1 to 4, wherein the tag is chosen from the group consisting of fluorescent dyes, enzymes, proteins, and radioactive labels.

6. The compound according to claim 5, wherein
the fluorescent dye is chosen from the group consisting of fluoresceins, rhodamines, BODIPY, Cy-3, Cy-5, and derivatives thereof.
the protein is chosen from the group consisting of peroxidases, luziferase, biotin, avidin, and antibodies or parts thereof;
the radioactive label is chosen from the group consisting of ^{99m}Tc, ¹⁸F, ¹¹C, ¹²⁴I, ¹²³I, ⁶⁴Cu²⁺, ⁶⁸Ga³⁺, ¹¹¹In³⁺, ¹⁴C, ³H, ³²P, and ³³P.

7. The compound according to claims 1 to 6, wherein the cyclic polyamine unit is cyclen with a structure of formula Ia or cyclam with a structure of formula Ib.

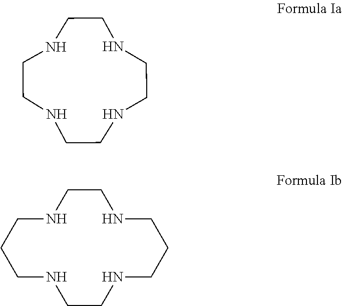

8. The compound according to claims 1 to 7, comprising two cyclic polyamine units that are connected via a bridging unit, that is selected from a group consisting of

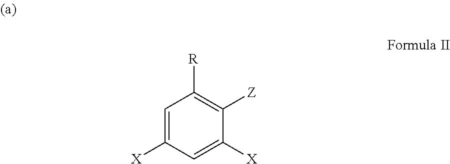

with
X=(CH₂)ₙ, (CH₂CH₂O), or CH₂OPhCH₂, or CO,
   wherein n=1, 2, or 3, and Ph=phenyl,
   wherein X is each covalently bound to a cyclic polyamine unit; and
Z=a halogen, a radiohalogen (such as 18F, ¹²³I), or H; and
R=H, or a tag as described in claim 4-6, preferably a fluorescent dye, ¹⁸F, ¹¹C, ¹²³I, or a labeled substituent,
wherein the tag is preferably a labeled substituent R₂,
with

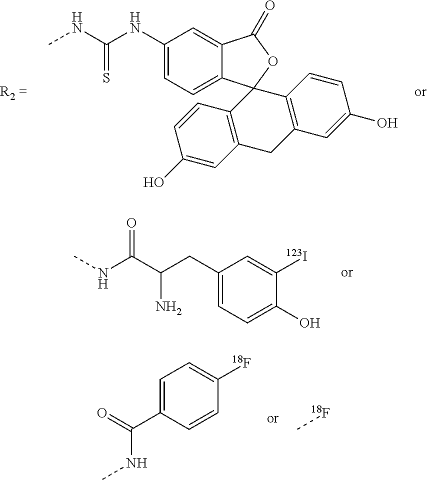

which optionally comprises a spacer R,, such that the labeled substituent R₂ with the spacer R₁ is

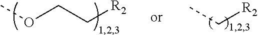

(b) a substituted benzene-based scaffold of formula III

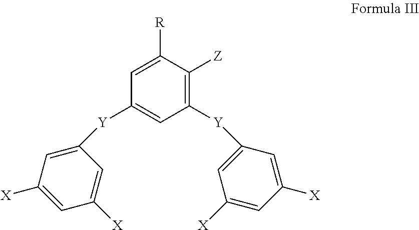

with
Y=(CH₂)ₙ with n=1, 2, or 3, (CH₂CH₂O)ₘ with m=0, 1, 2, or 3 O(CH₂)ₚO (with p=1, 2, 3, or 4), or O (independently of each other), or mixtures thereof; and
Z=a halogen, a radiohalogen (such as ¹⁸F, ¹²³I), or H; and
R and X as described for formula II above;

(c) an adamantane-based scaffold of formula IV

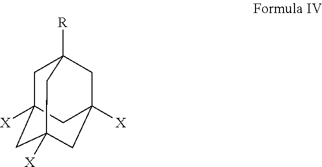

with X and R as described above for formula II;

(d) a peptide-based scaffold of formula V (peptidic scaffolds with αamino acids) or formula Va (peptidic scaffolds with β-amino acids)

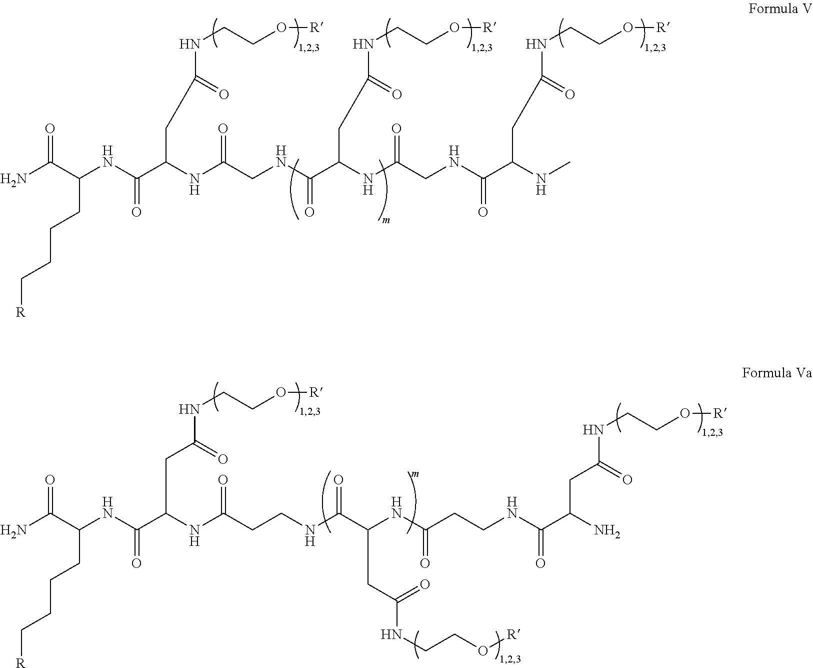

wherein m=1, 2, 3, 4, 5, or 6, and
with R′=an alkyl or aryl bridge connected with a cyclen or a cyclam,
independently of each other; and
with R as described for formula II.

9. The compound according to claims 1 to 8, as shown in table 1 or table 2, or of formula VI, VII, or VIII.

10. Use of a compound according to claims 1 to 9 for binding phosphatidylserine.

11. Use of a compound according to claims 1 to 9 for apoptosis imaging.

12. Use according to claim 10 or 11, using positron emission tomography (PET), single-photon emission computed tomography (SPECT), magnetic resonance imaging (MRI), or optical imaging.

13. Use according to claims 10 to 12, for assessing the status of a tumor or the response of a patient suffering from a tumor, from an infection, and/or from an inflammation, to an anti-tumor therapy, to anti-infection therapy and/or to anti-inflammatory therapy, respectively.

14. A method for detecting the progression of a tumor, of an infection, and/or of an inflammation in a tissue of a patient, comprising
a) administering a compound according to claims 1 to 9 to a patient, and
b) measuring the amount or the concentration of the compound in the tissue.

15. A method for synthesizing of a compound according to claims 1 to 9, wherein at least two cyclic polyamine units are covalently bound to each other via a bridging unit.

16. The method according to claim 15, wherein the bridging unit is selected from the formulas II, III, IV, V, Va.

17. A pharmaceutical composition, comprising a compound of claims 1 to 9.

18. The pharmaceutical composition according to claim 17, further comprising a suitable carrier and/or additive.

19. A kit for binding phosphatidylserine, comprising a compound of claims 1 to 9, and optionally buffers for detecting phosphatidylserine in vitro.

20. Use of a compound according to claims 1 to 9 or pharmaceutical composition according to claim 17 for in-vivo or in-vitro detection of apoptosis.

TABLE 1
Preferred compounds of the invention: Each compound shown comprises at least two cyclic polyamine units in the form of a cyclen or cyclam, joined by a bridging unit. The bridging unit may also carry
a detectable tag R, which may be a labeled substituent R₂ that can, optionally, comprise a spacer R₁. Any given bridging unit given below can be combined with any combination of R₁ and R₂ written in the same
portion of the table (separated by bold lines). The preferred compounds are shown here complexed with Zn²⁺. It is possible, however, that the compounds form a complex with any other multivalent
(in particular a bi- or trivalent) metal ion as described herein, or with mixtures thereof.

| Cyclic polyamine units joined by a | R | |
|---|---|---|
| bridging unit | Linker (L) | Tag (T) = R2 |
| 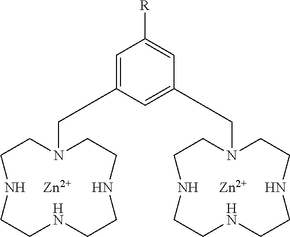 | 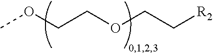 | 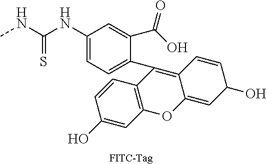 |
| | 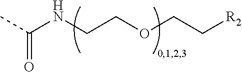 | 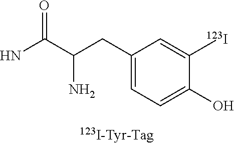 |
| | | 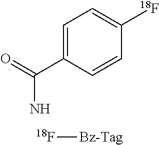 |
| | | ¹⁸F |
| 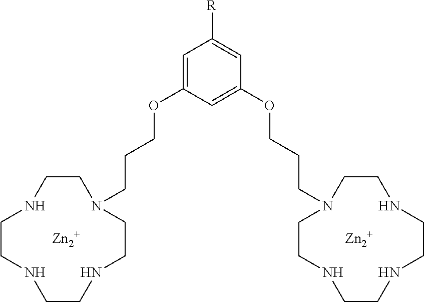 | 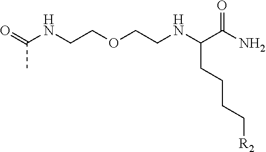 | ¹⁸F FITC-Tag ¹²³I-Tyr-Tag ¹⁸F—Bz-Tag |
| | 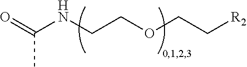 | |
| 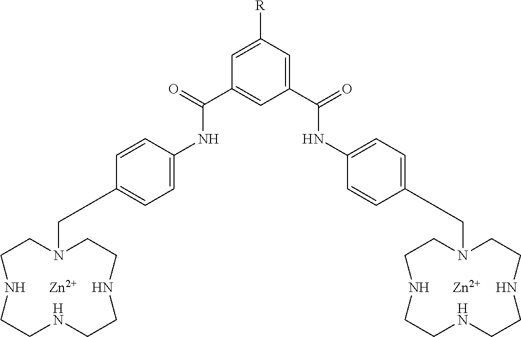 | 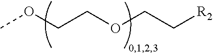 | ¹⁸F FITC-Tag ¹²³I-Tyr-Tag ¹⁸F—Bz-Tag |
| | 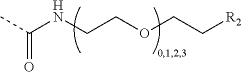 | |
| 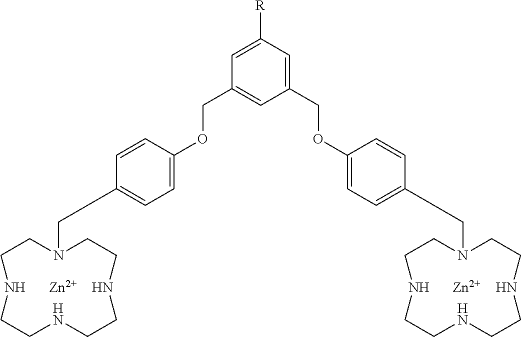 | 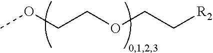 | ¹⁸F FITC-Tag ¹²³I-Tyr-Tag ¹⁸F—Bz-Tag |
| 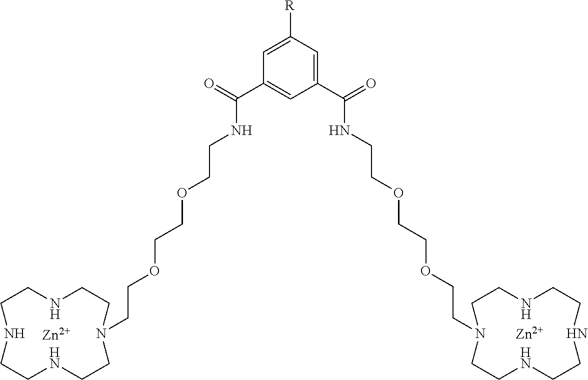 | 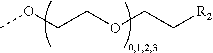 | ¹⁸F FITC-Tag ¹²³I-Tyr-Tag ¹⁸F—Bz-Tag |
| 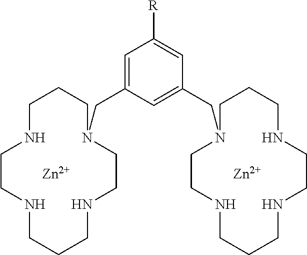 | 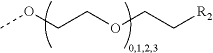 | ¹⁸F FITC-Tag ¹²³I-Tyr-Tag ¹⁸F—Bz-Tag |
| 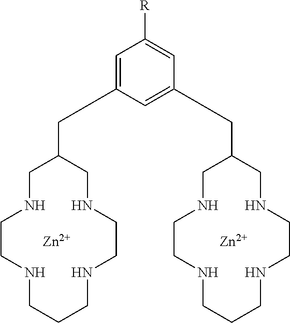 | 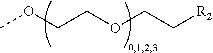 | ¹⁸F FITC-Tag ¹²³I-Tyr-Tag ¹⁸F—Bz-Tag |
| 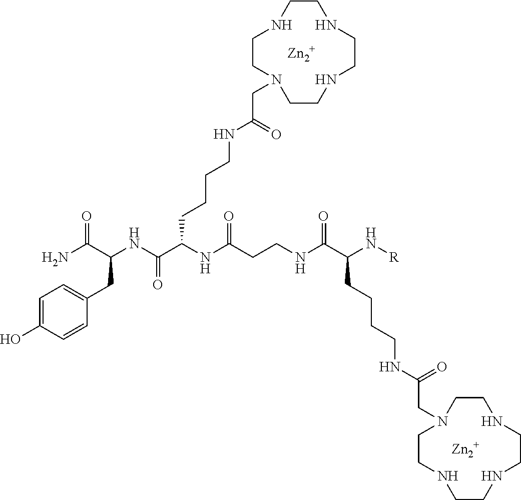 | 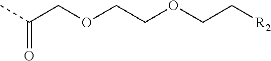 | ¹⁸F FITC-Tag ¹²³I-Tyr-Tag ¹⁸F—Bz-Tag |
| 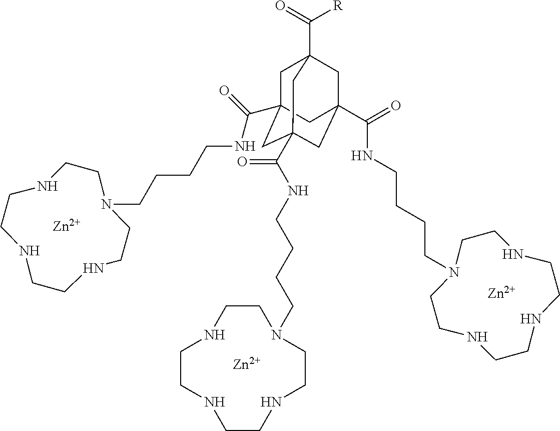 | 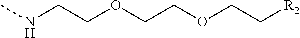 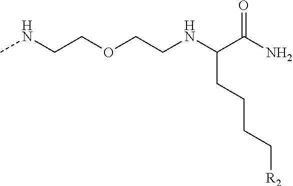 | ¹⁸F FITC-Tag ¹²³I-Tyr-Tag ¹⁸F—Bz-Tag |
| 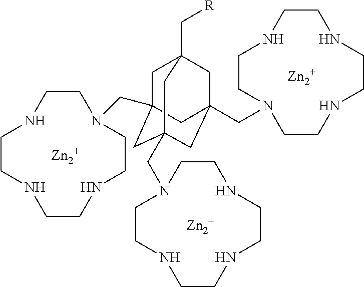 | 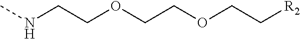 | ¹⁸F FITC-Tag ¹²³I-Tyr-Tag ¹⁸F—Bz-Tag |
| 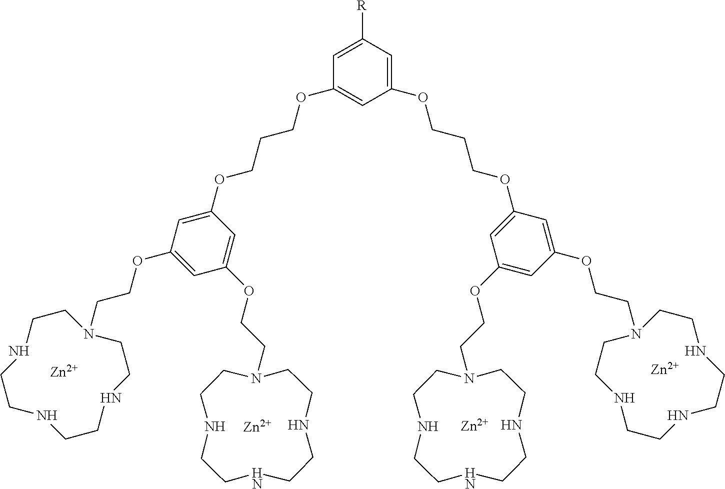 | 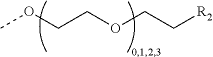 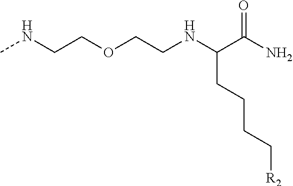 | ¹⁸F FITC-Tag ¹²³I-Tyr-Tag ¹⁸F—Bz-Tag |
| 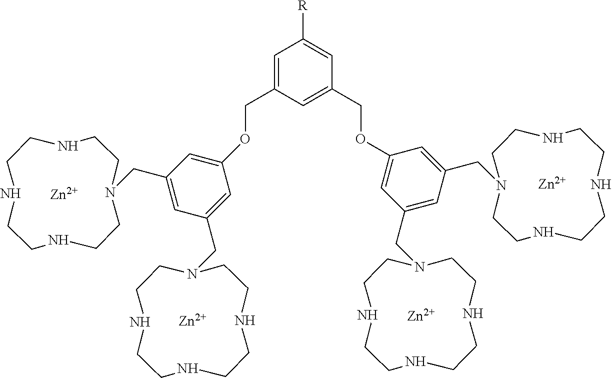 | 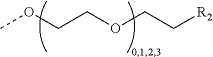 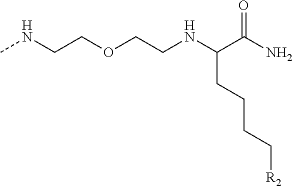 | ¹⁸F FITC-Tag ¹²³I-Tyr-Tag ¹⁸F—Bz-Tag |
| 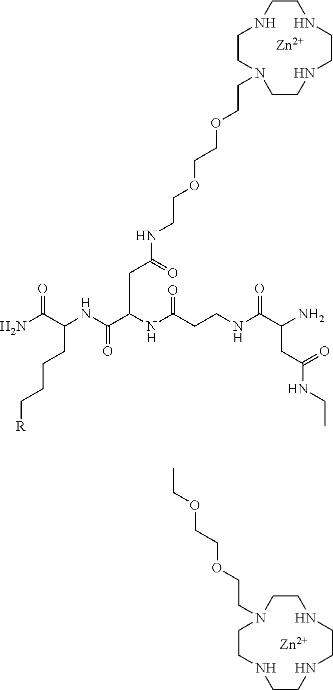 | | ¹⁸F FITC-Tag ¹²³I-Tyr-Tag ¹⁸F—Bz-Tag |
| 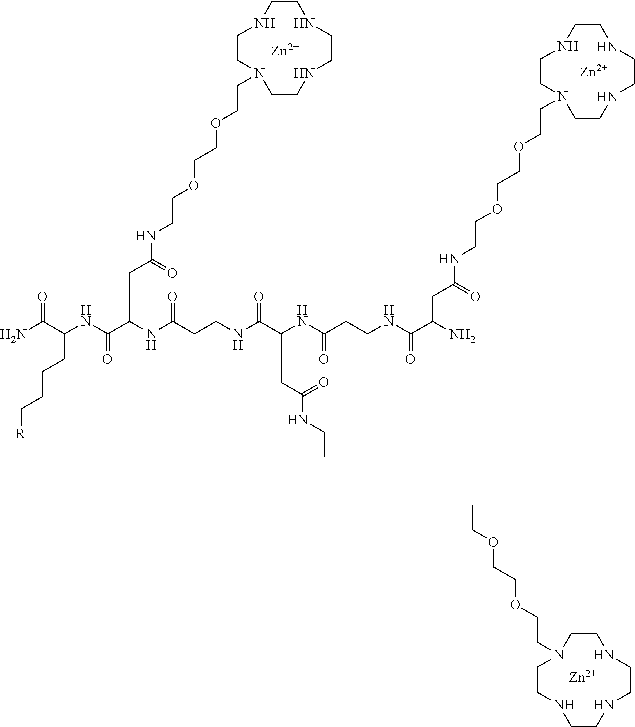 | | ¹⁸F FITC-Tag ¹²³I-Tyr-Tag ¹⁸F—Bz-Tag |
| 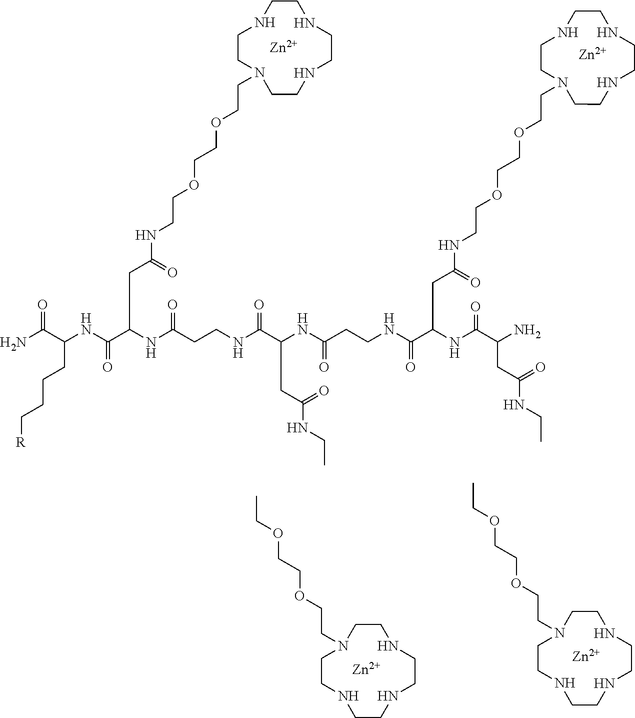 | | ¹⁸F FITC-Tag ¹²³I-Tyr-Tag ¹⁸F—Bz-Tag |

TABLE 2
Preferred compounds of the invention: Each compound shown comprises at least
two cyclic polyamine units in the form of a cyclen or cyclam, joined by a
bridging unit. The bridging unit may also carry a residue A. Z is a radiohalogen
for detecting the compound. The cyclic polyamine unit joined by the bridging
unit given below can be combined with any combination of residue A and Z in the table.
The preferred compounds are shown here complexed with Zn²⁺. It is possible, however, that
the compounds form a complex with any other multivalent (in particular a bi- or trivalent)
metal ion as described herein, or mixtures thereof.

| Cyclic polyamine units joined by a | | |
|---|---|---|
| bridging unit | A | Z |
| 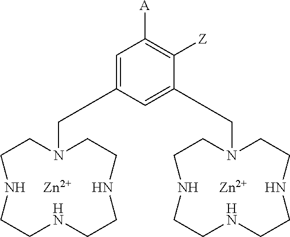 | OH, alkyloxy, aryloxy | ¹²³I, ¹²⁴I, ¹⁸F |

### EXAMPLES

### a) Synthesis of Fluorescence-Labeled Bis-Cyclen Complexes

a-1

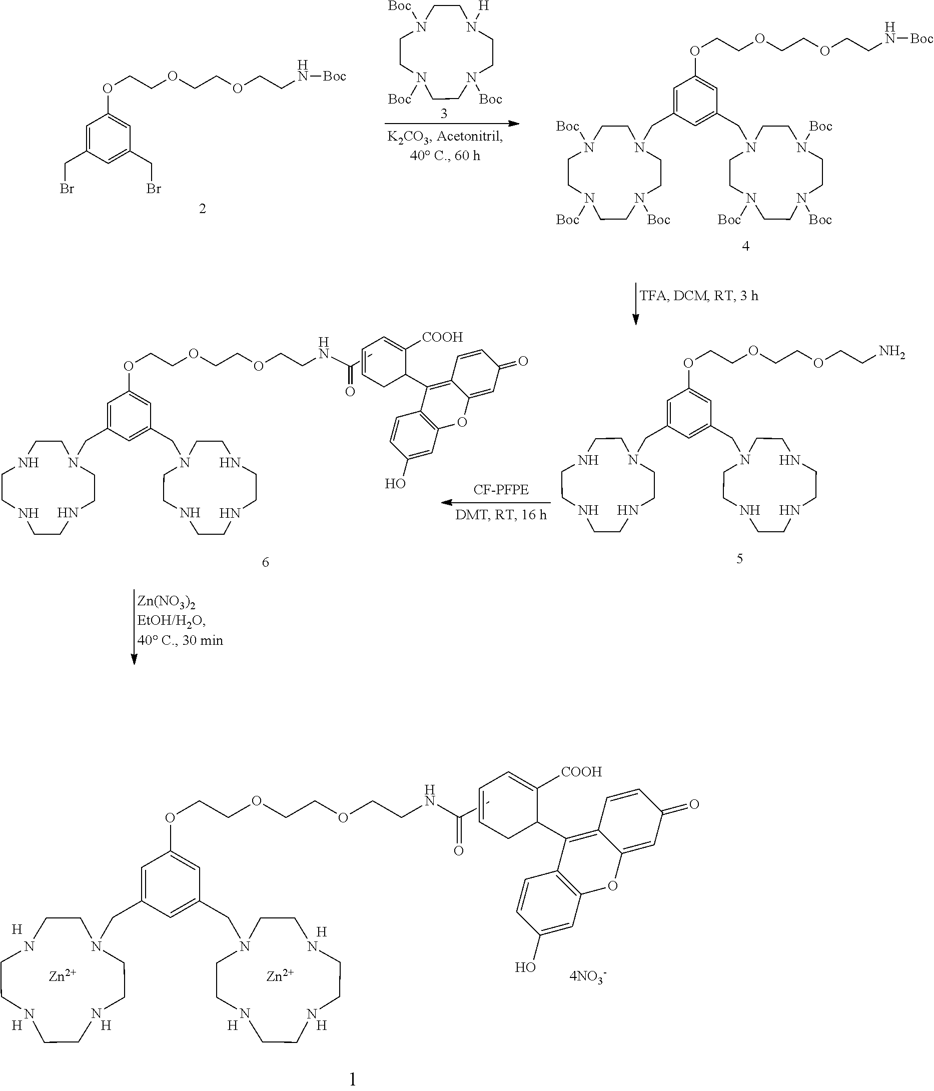

All solvents used were bought as water-free p. A.-solvents. Silica gel 60 (0.04 - 0.063 μm, Fluka) was used for the column chromatography purification. The preparative HPLC-purification was performed with an HPLC by Agilent (1100 Series) using the semi-preparative column Chromolith® SemiPrep, RP18e, 100-10 mm (Merck).

### Synthesis of Compound 4

The bromo compound 2 (22) (130 mg, 254 μmol) and 1,4,7-tris(tert-butyloxycarbonyl)cyclen (3) (23) (295 mg, 624 μmol, 2.5 eq.) were dissolved in 4 ml acetonitril and potassium carbonate (175 mg, 1.27 mmol) was added. The suspension was stirred for 60 h at 40° C. Subsequently, the potassium carbonate was removed by filtering and the solvent was removed i. vac. Following column chromatography purification using silica gel with EtOAc/petrol ether=1:1 as eluent, a white solid substance was obtained.

### Yield: 220 mg (90%)

### Synthesis of Compound 5

The biscyclen compound 4 (220 mg, 170 μmol) was dissolved in 1.4 ml 50% TFA in dichloromethane and stirred for 3 h at room temperature. The solvent was removed i. vac. No further purification was performed.

Yield: quantitatively

### Synthesis of Compound 6

The amine 5 (8.3 mg, 13.9 μmol) was dissolved in 250 μl DMF anhydr. 5-/6-carboxyfluoresceinpentafluorophenylester (24) (7.5 mg, 13.9 μmol) was also dissolved in DMF anhydr. and added to 5. The reaction mixture was stirred for 16 h at room temperature. The solvent was removed i. vac. The raw product was purified at the semi-preparative HPLC (A=H₂O, B=ACN, 0.1% TFA; 5 min 40% B).

MALDI-MS: m/z=968.6 (M+H⁺), 990.6 (M+Na⁺) (calc 967.55)

Synthesis of Compound 1

The ligand 6 (1.1 mg, 1.16 μmol) was dissolved in 20 μl EtOH and 23.1 μl of an aqueous 0.1 mol/l Zn(NO₃)₂-solution (2.31 μmol). The solution was heated for 40 minutes to 50° C. After lyophilizing a yellow solid substance was obtained.

### Yield: 100%

### a-2 Flow Cytometry (FACS)

### a-2.1 Flow Cytometry Methodology

Flow cytometry is a technology for counting and examining cells which pass in a flow of single cells through a laser beam. The laser light scatter provides information about cell features. The forward scatter (FSC) depends basically on the size of the cell, whereas the side scatter (SSC) is used to measure the granularity of the cell. Furthermore, if the cells carry fluorescence dyes, the respective fluorescence signal can be detected.

Flow cytometry is a widely applied technique in cell biology. Especially in the research of cell death mechanisms it is a standard procedure to count and differentiate between apoptotic, necrotic and healthy cells. For the detection of apoptosis the protein Annexin V is applied which is usually attached to a green fluorescence dye. Because Annexin V can also bind to necrotic cells, the red fluorescent nuclear stain propidium iodide is simultaneously applied as a necrosis marker.

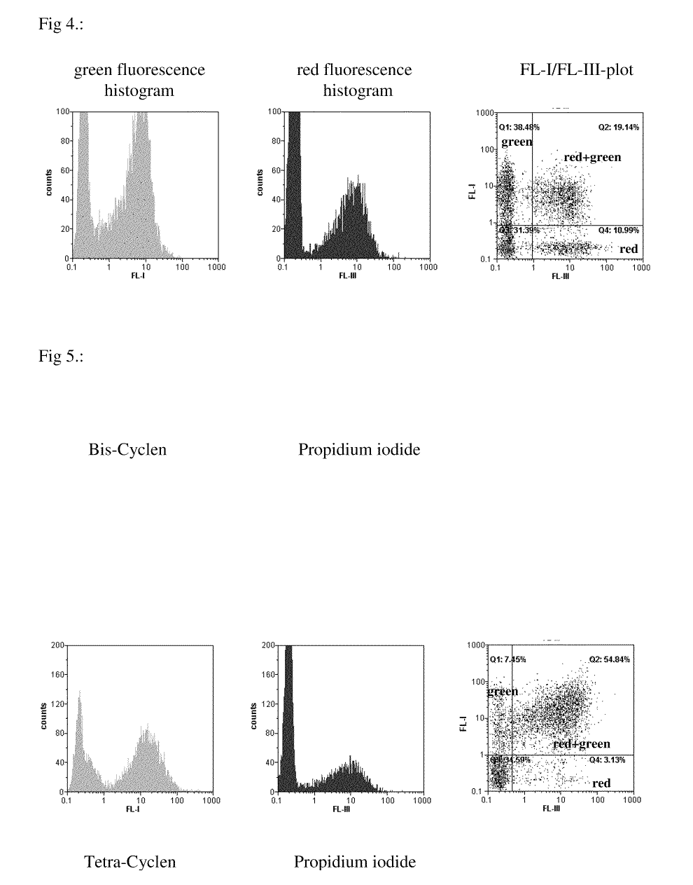

The resulting fluorescence diagrams that are obtained from a flow cytometry procedure with Annexin V and propidium iodide are shown below. The detection channel for green fluorescence is termed FL-I (FIG. 4), the one for red fluorescence FL-III (FIG. 4). The plot of cell number (counts) versus FL-I intensity is called histogram. The fluorescence signal of the sample is always accompanied by a second signal at low intensities (left in the histogram) which is background fluorescence. By plotting the red fluorescence versus the green fluorescence (FL-I/FL-III-plot) a diagram is obtained in which the cells can be divided into red, green and red+green fluorescent cells. After gating the signals into four quadrant regions (Q1-Q4) (FIG. 4), the percentages of the differently stained cells is indicated in the diagram.

### a-2.2 Flow Cytometry Analysis

Both the fluorescein-labeled bis-cyclen compound and the tetra-cyclen compound stained cells which were treated with staurosporine in order to induce apoptosis (FIG. 5). The green fluorescence signal indicates the number of cells that interact with these cyclen compounds. The FL-I/FL-III-plot shows that the majority of stained cells are not only green but also red fluorescent which means that the cells carry additionally to the cyclen compounds (green) also the necrosis marker propidium iodide (red).

FACS measurements were performed with a DAKO Galaxy Flow Cytometry System using the following experimental setup: Jurkat-cells were grown to a density of about 1*10⁶ cells/ml. Apoptosis was induced by adding 1 μmol/l staurosporine (Stepczynska A et al, Staurosporine and conventional anticancer drugs induce overlapping, yet distinct pathways of apoptosis and caspase activation, Oncogene (2001) 20, 1193). After incubation for 4 h at 37° C., the cells were washed with cold PBS-buffer (140 mmol/l NaCl, 10 mmol/l KCl, 6.4 mmol/l Na₂HPO₄, 2 mmol/l KH₂PO₄) and resuspended in cold TES-buffer (5 mmol/l TES, 145 mmol/l NaCl). 10⁶ cells in 100 μl buffer with 1 μl of a 0.5 mmol/l aqueous solution of complex 1 were incubated for 15 minutes at 37° C. Subsequently, cells were washed twice with 500 μl TES-buffer and the cells were resuspended in 400 μl TES-buffer. 100 μl of the probe were diluted with 900 μl of buffer and measured immediately. To discriminate against necroses, 10 μl propidiumiodide solution (250 μg/ml) were added directly before the measurement.

TABLE 2
Examples for apoptosis detection by Annexin V and three
different Zn(II)-complexes on staurosporine treated Jurkat
cells, using propidium iodide (PI) for necrosis discrimination.

| | | | % necrosis/ |
|---|---|---|---|
| | % apoptosis | % necrosis | late apoptosis |
| Apoptosis Sensor | (green) | (PI) (red) | (red + green) |
| Anx V | 22 | 18 | 25 |
| bis-Zn(II)-Cyc A | 32 | 25 | 15 |
| bis-Zn(II)-Cyc B | 34 | 24 | 18 |
| tetra-Zn(II)-Cyc | 30 | 25 | 13 |

Examples for apoptosis detection with Annexin V and three different Zn(II)-complexes on staurosporine treated Jurkat cells, using propidium iodide (PI) for necrosis discrimination are summarized in Table 2. The results demonstrate the parallelism of apoptosis-detection potency of the three Zn(II)-Cyclene derivatives with Annexin V.

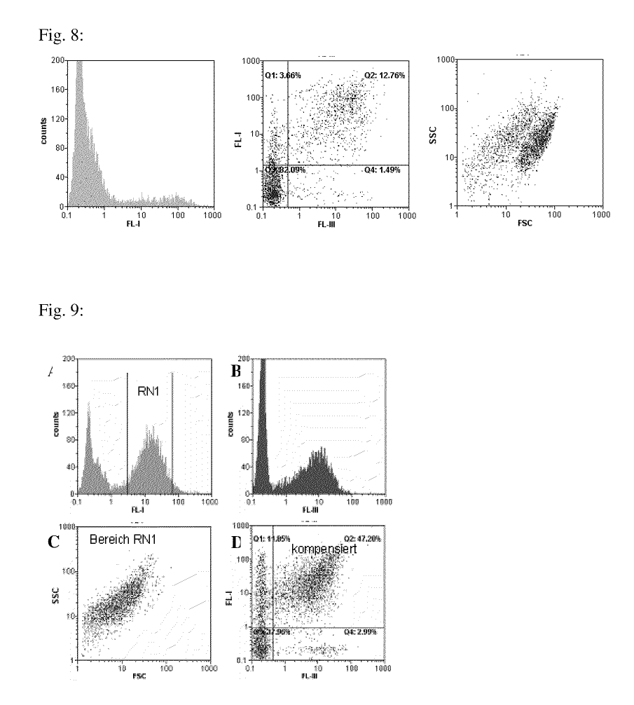

FACS analysis of un-induced Jurkat cells showed very little apoptosis and thus very little binding of the bis-cyclen compounds to vital living cells. FIG. 8 shows the results for compound 1, the vital cells are the largest fraction with 82% in Q3, few apoptotic cells (4%) in Q1, necrotic cells in Q2 and Q4 (together 14%). But no labelling was seen of the vital living cells in Q3 with compound 1. The same results were obtained for compound xx. If apoptosis is induced via staurosporin (FIG. 9), labeling of the apoptotic cells (Q1=12%) and necrotic cells (Q2=42%) with compound 1 can be observed (same results with compound xx).

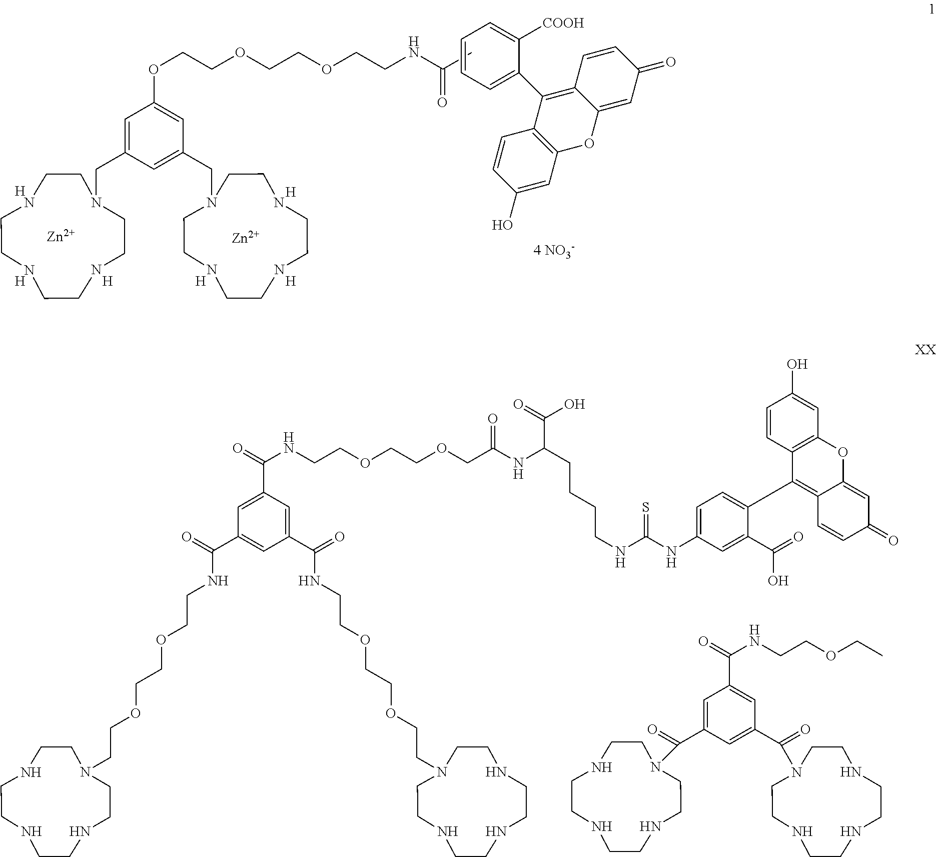

In a direct comparison with Annexin-V, it was shown that bis-cyclen showed labeling of some apoptotic and most necrotic cells. FIG. 10 shows for example compound AB labeling apoptotic cells in Q2 and necrotic cells in Q4, while Annexin-V labels apoptotic cells in Q1 and Q2.

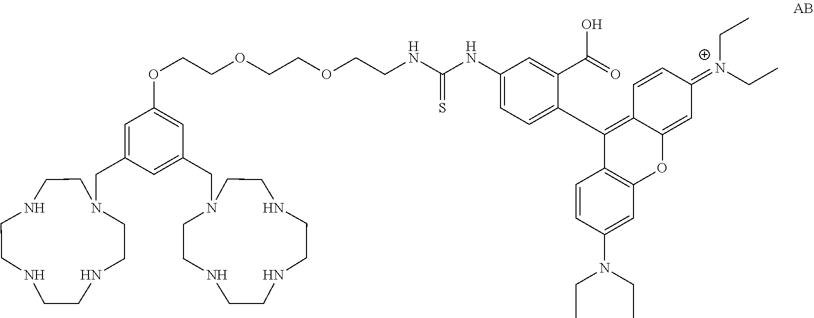

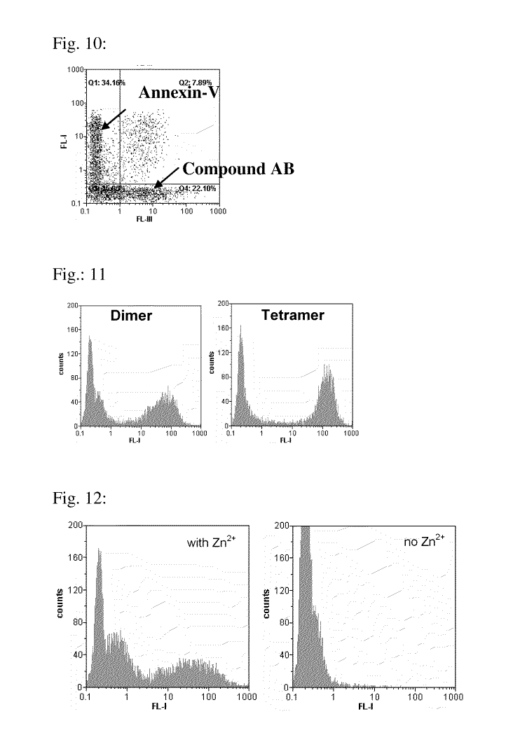

It could be observed with induced apoptotic Jurkat cells, that multimerisation of bis-cyclen compounds led to an increase in labelling intensity while the percentage of labelled cells remained unchanged. FIG. 11 shows an example for compound 1 (dimer=two cyclen-units) and compound we (tetramer=four cyclen-units) where the median fluorescence intensity for the tetramer is 130, while the median fluorescence intensity for the dimer is 56 while the percentage of labelled cells remained unchanged.

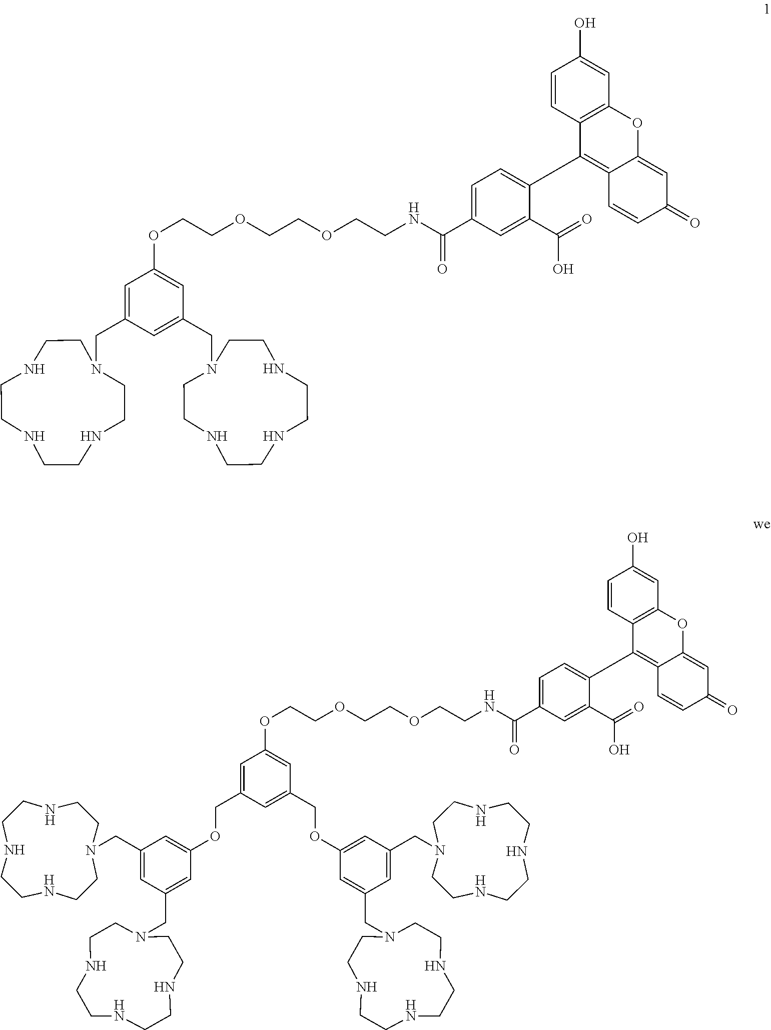

The complexation of bivalent cations, especially with Zn²⁺, is important for the binding, specificity of the bis-cyclen compounds. This is, e.g. very pronounced for compound 1, which shows as a complex with Zn²⁺ good binding to induced Jurkat cells, but almost no binding to induced Jurkat cells if no Zn²⁺ is complexed (see FIG. 12). The same effect was observed with compound we although not as pronounced as with compound 1.

In order to verify the FACS data with visual data confocal microscopy was performed. Confocal microscopy was also performed on Jurkat cells and apoptosis was induced by incubating 10⁶ cells/ml in 1 μM staurosporin for 4 h (Stepczynska A et al, Staurosporine and conventional anticancer drugs induce overlapping, yet distinct pathways of apoptosis and caspase activation Oncogene (2001) 20, 1193). The experiments were also carried out in TES-buffer. For compound binding 10⁶ cells (either induced with staurosporin or non-induced) were incubated for 15 min at RT in 100 μl cold TES buffer with a final concentration of 5 μM compound, then the cells were fixed onto polylysine coated slides and shortly incubated with propidium iodine (PI).

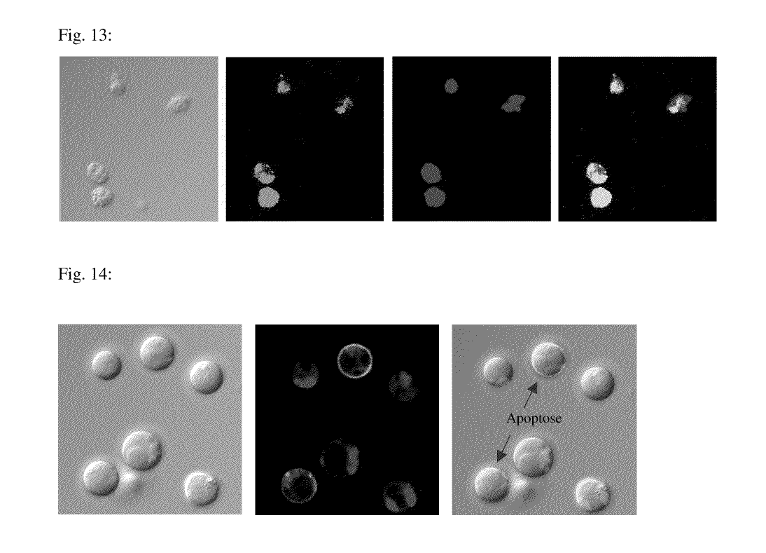

The incubation of induced cells with compound 1 and PI showed good uptake of compound 1 and PI into to same cells, showing the uptake of compound 1 into necrotic cells (FIG. 13).

A co-incubation of induced cells with Annexin-V and compound AB was performed to visualise apoptotic cells. FIG. 14 shows the green membrane staining of Annexin-V for apoptotic cells. The red labeling from compound AB co-labels the apoptotic cells stained with Annexin-V but also shows labeling of cells, which were not detected by Annexin-V. This clearly shows labeling of apoptotic and necrotic cells with compound AB.

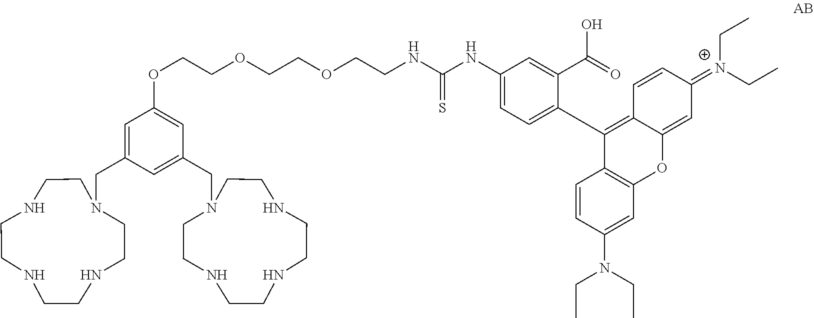

### b) Synthesis of Radioactively-Labeled Bis-Cyclen Complexes

### b-1 Synthesis of Compound 15

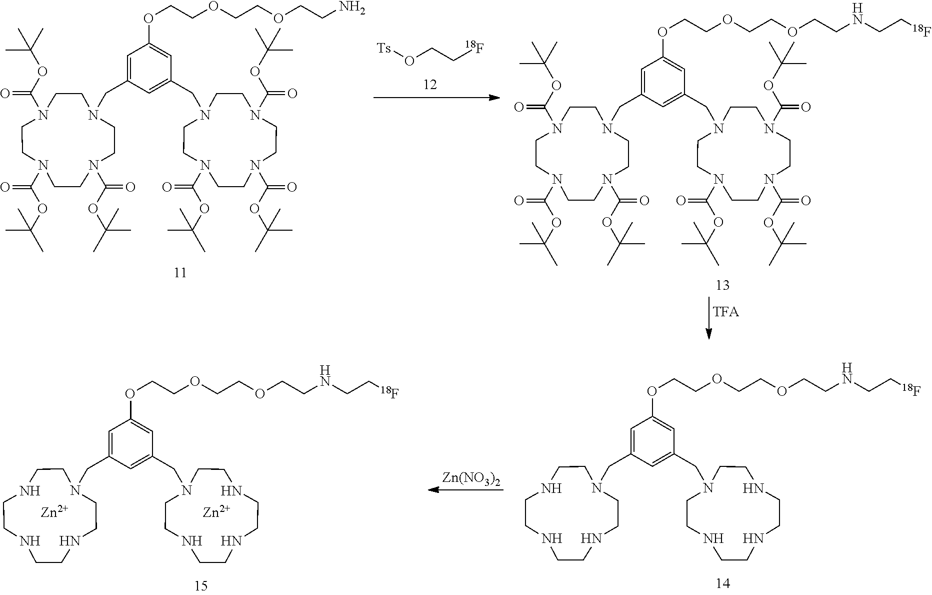

Synthesis of the 2-[¹⁸F]fluoroethyltosylat 12 was performed according to the literature (25). The ¹⁸F-Fluorethyltosylate was dissolved in 200 μL anhydrous DMF and 5 mg of K₂CO₃ was added. The precursor 11 (2 mg) in 10 μL anhydrous DMF was added and the solution was heated for 30 minutes at 120° C. The reaction mixture was run over an RP18 HPLC. Solvent was evaporated. Radiochemical yield: 65%

The ¹⁸F-fluorethoxy-bis(cyclen) was dissolved in 50 μL ACN and 200 μl TFA was added. After 10 min at RT the solvent was removed. The reaction mixture was run over an RP18 HPLC. The deprotected ¹⁸F-fluorethoxy-bis(cyclen) was dissolved in 100 μL PBS-buffer, 10 μL 1.5 mM Zn(NO₃)₂-solution was added and the solution was heated for 5 min at 70° C. The mixture was diluted with 400 μL PBS. Radiochemical yield>50% (d.c)

### b-2 Synthesis of Compound 19

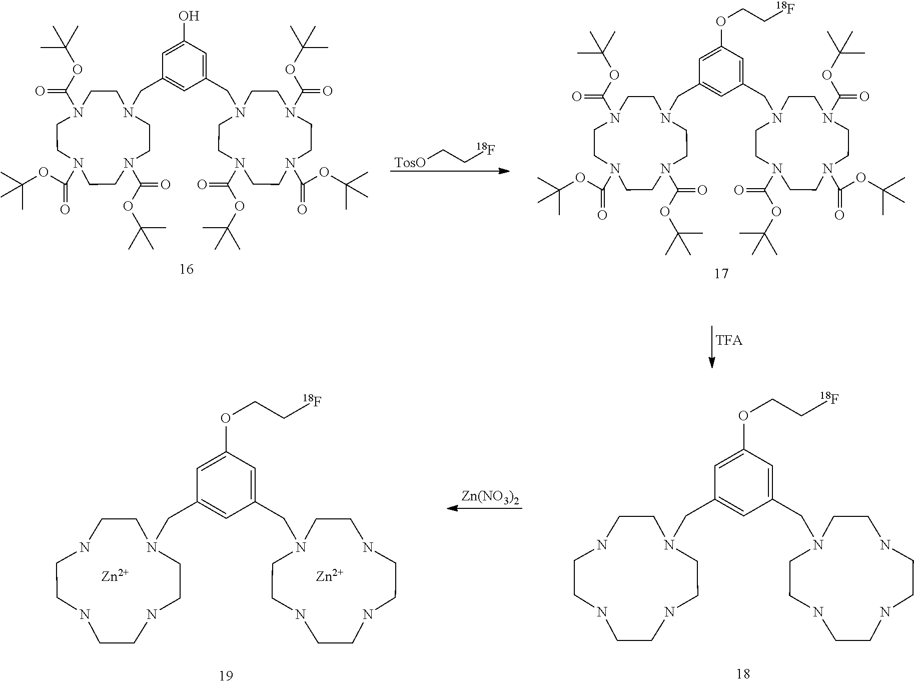

¹⁸F-Fluorethyltosylate was dissolved in 200 μL anhydrous DMF and 5 mg of K₂CO₃ was added. The phenolic precursor 16 (2 mg) in 10 μL anhydrous DMF was added and the solution was heated for 35 minutes at 120° C. The reaction mixture was purified by preparative HPLC: Chromolith® SemiPrep RP-18e column (100×10 mm, Merck, Darmstadt, Germany), gradient,100% Water (0.1%TFA) to 100% acetonitrile (0.1% TFA), flow: 8 mL/min; (t_{R}=5.7 min). Radiochemical yield: 77%

The ¹⁸F-fluorethoxy-BOC-bis(cyclen) 17 was dissolved in 50 μL MeCN and 200 μL TFA were added. After 10 min at RT the solvent was removed in vacuum. The reaction mixture was purified by preparative HPLC: Chromolith® SemiPrep RP-18e column (100×10 mm, Merck, Darmstadt, Germany), gradient, 100% Water (0.1%TFA) to 100% acetonitrile (0.1% TFA), flow: 8 mL/min; (t_{R}=1.7 min). The quantitatively deprotected ¹⁸F-fluorethoxy-bis(cyclen) was dissolved in 100 μL PBS-buffer, 10 μL 1.5 mM Zn(NO₃)₂-solution was added and the solution was heated for 5 min at 70° C. The mixture was diluted with 400 μL PBS and used without further purification.

### b-3 Biodistribution and PET Diagnostic

The biodistribution was performed in male Balb/c mice (6-8 weeks, body weight approx. 20 g). Hepatic apoptosis were induced by intraperitoneal injection of Cycloheximid (50 μg/kg in 500 μL PBS) (J. F. Tait, C. Smith, Z. Levashova, B. Patel, F. G. Blankenberg, J. Vanderheyden, J. Nucl. Med. 2006, 47, 1546). 1.5 h and 3.5 h after induction of hepatic apoptosis the 18F-labeled compound was injected i.v. (1-2 MBq in 100 μL PBS). The organ distribution was done after 30 min, 1 h, 2 h and 3 h. The amount of 18F-compound was determined by gamma counter.

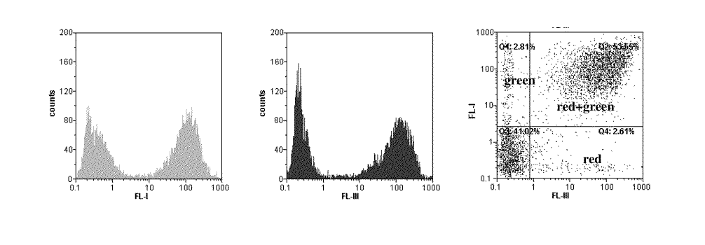

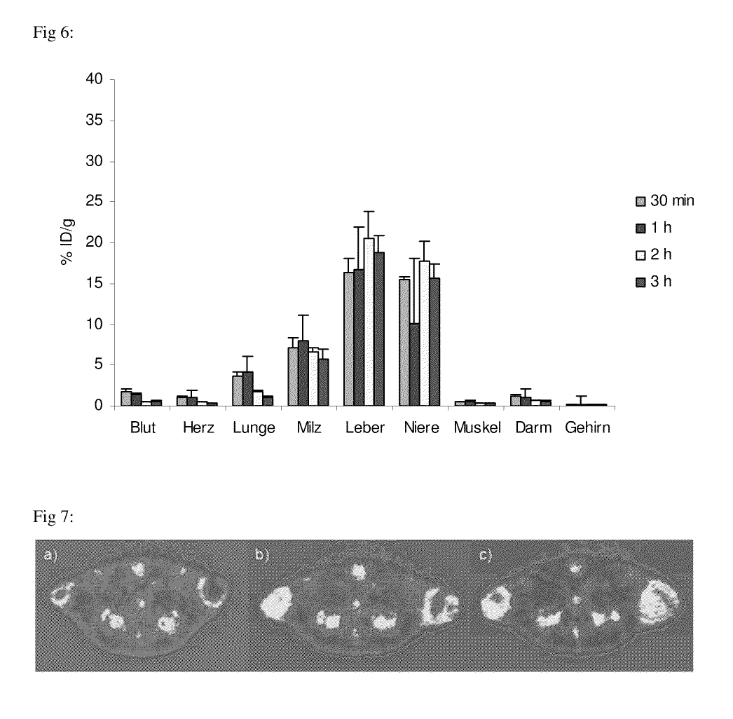

Biodistribution data of compound 19 for the 3.5 h time point after induction of hepatic apoptosis is shown in FIG. 6. The compound showed accumulation in the apoptotic liver as early as 30 min after injection and the signal was stable for more than 3 h.

The PET images were obtained using a radiation model. Male Copenhagen rats with approx. 180 g body weight were transplanted with the hormone independent adenocarcinoma (Prostata) R3327-ATI on the left and right upper leg. The left tumor, as it reached a size of 3.5 cm³ was radiated by linear particle accelerator with a single dosis of 50 Gy, whereas the right tumor served as control.

One day prior to the radiation as well as 6 and 10 d after radiation, the 18F-compound 19 (15-25 MBq in ca. 200 μL PBS) was injected i.v. and PET-aquisition with 1 h dynamic PET measurements were performed. FIG. 7 clearly showed an increase in accumulation of compound 19 on day 6 and day 10 for the irradiated tumor in comparison to the control tumor.

### c) Synthesis of Radioactively-Labeled Tetra-Cyclen Complexes

### c-1 Indirect Labeling, Compound 21:

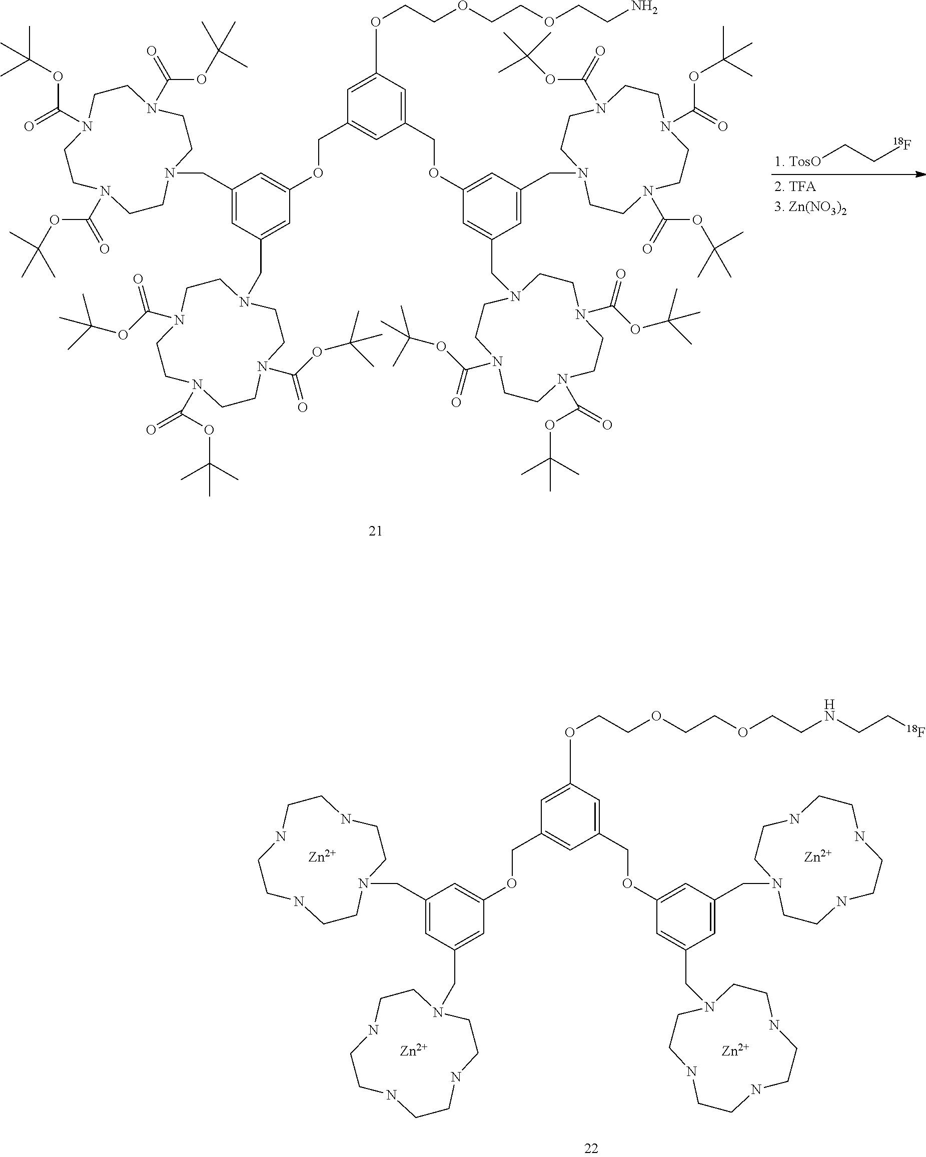

The radiolabeling was done in according to compound 15 and 19 to give compound 22 with an overall radiochemical yield of 30% corrected for decay regarding [¹⁸F]fluoro ethyl tosylate.

### c-2 Direct Labeling, Compound 17:

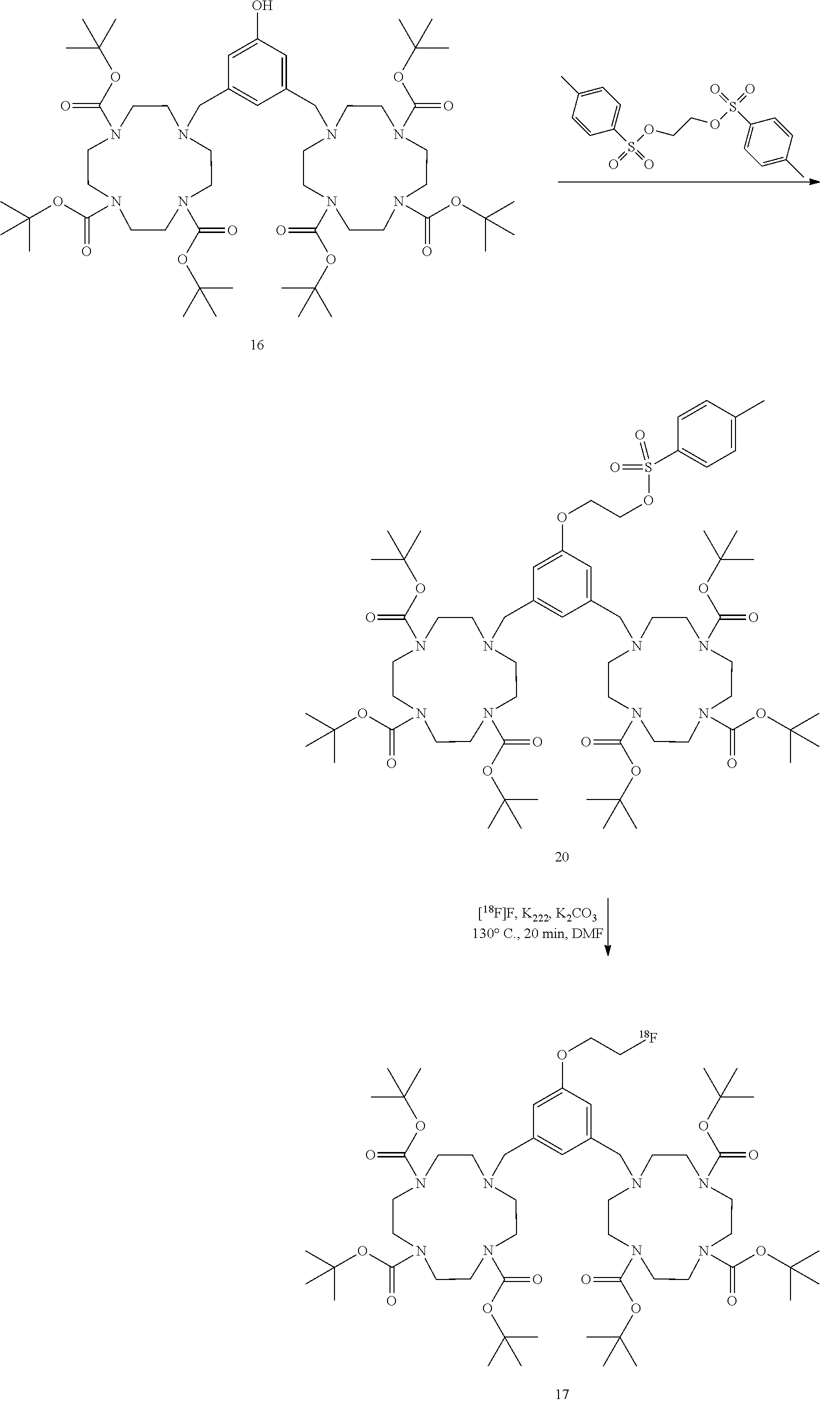

### Synthesis of Compound 20

Compound 16, (25 mg, 24 μmol) was dissolved in 100 mL extra dry MeCN and potassium carbonate (10 mg, 72 μmol, 3 eq.) and ethylene glycol ditosylate (100 mg, 235 μmol, 10 eq.) were added. The reaction mixture was refluxed for 16 h. Potassium carbonate was filtered off and the solvent was removed in vacuum. The crude was purified via semipreparative HPLC (100% H2O (0.1% TFA) to 80% MeCN in 0.1 trifluoroacetic acid in 8 min). After removing the solvent in vacuum 19 mg of product 20 were obtained as a colorless solid in 67% yield.

Analytical HPLC: Chromolith® Performance RP-18e column (100×4.6 mm; Merck KGaA, Darmstadt, Germany); solvent gradient: 100% water (0.1% trifluoroacetic acid) 95% acetonitril in 0.1% trifluoroacetic acid in 5 min., flow: 4 mL/min Rt (analyt. HPLC, λ=215, 254 nm): 4.2 min
1H-NMR (500 MHz, DMSO-D6, 343 K): δ=7.80-7.77 (m, 2 H, H-3, 3J2,3 =8.2 Hz); 7.49-7.45 (m, 2 H, H-3); 6.84-6.67 (m, 3 H, H-9, H-11); 4.35-4.31 (m, 2 H, H-7); 4.17-4.13 (m, 2 H, H-6); 3.74 (bs, 4 H, H-12); 3.49 (bs, 8 H, H-Cy4); 3.37-3.27 (m, 16 H, H-Cy3, H-Cy2); 2.60 (bs, 8 H, H-Cy1); 2.42 (s, 3 H, H-1); 1.41 (s, 18 H, H-Boc3); 1.36 (s, 36 H, H-Boc3) ppm.
13C-NMR (500 MHz, DMSO-D6, 343 K): δ=157.48 (C-8); 155.31 (C-Boc1); 154.30 (C-Boc1); 144.42 (C-5); 139.53 (C-2); 137.47 (C-10); 129.64 (C-3); 127.08 (C-4); 125.50 (C-11); 114.94 (C-9); 78.05 (C-Boc2); 77.84 (C-Boc2); 68.43 (C-7); 65.20 (C-6); 53.40 (C-12); 48.28 (C-Cy1, C-Cy3); 47.03 (C-Cy2); 46.20 (C-Cy4); 27.86 (C-Boc3); 27.64 (C-Boc3); 20.59 (C-1) ppm.

### Direct Radiosynthesis of Compound 17

Aqueous [18F]Fluoride (1 GBq) was trapped on a QMA cartridge (Waters) and eluted with 5 mg K2.2.2 in 0.95 ml MeCN+1 mg K2CO3 in 50 μl water into a Wheaton V-vial. The solvent was removed by heating at 120° C. for 10 min under a stream of nitrogen. Anhydrous MeCN (1 mL) was added and evaporated as before. A solution of precursor 20 (5 mg) in 500 μL anhydrous DMF was added. After heating at 130° C. for 30 min the crude reaction mixture was mixed with 3 mL H2O and purified by preparative HPLC: Chromolith® SemiPrep RP-18e column (100×10 mm, Merck, Darmstadt, Germany), gradient,100% Water (0.1% TFA) to 100% acetonitrile (0.1% TFA), flow: 8 mL/min; tR=5.7 min. The collected HPLC fraction was diluted with 40 ml water and immobilized on a Sep-Pak light C18 cartridge (Waters), which was washed with 5 ml water and eluted with 2 ml ethanol to deliver 340 MBq product (50%, corrected for decay; radiochemical purity>95% TLC, >95% HPLC) in a overall synthesis time of 60 min. The desired F-18 labeled product was analyzed using analytical HPLC: Chromolith® Performance RP-18e column (100-4.6 mm; Merck KGaA, Darmstadt, Germany); solvent gradient: start 100% water (0.1% trifluoroacetic acid)-95% acetonitril in 0.1% trifluoroacetic acid in 5 min., flow: 4 mL/min and confirmed by co-injection with the corresponding non-radioactive F-19 fluoro-standard on the analytical HPLC.

### ABBREVIATIONS

ACN—Acetonitril
CF-PFPE—5-/6-Carboxyfluoresceinpentafluorphenylester
DCM—Dichloromethane
DMF—N,N-Dimethylformamide
EtOAc—Ethyl acetate
EtOH—Ethanol
FACS—Fluorescence-activated cell sorting
FITC—Fluorescein
TES—[N-[Tris(hydroxymethyl)methyl]-2-aminosulfonic acid]
PI—Propidium iodide
TFA—Trifluoro acetic acid
   References 1. Blankenberg F G, Katsikis P D, Tait J F et al. Increased localisation of Tc-99m hydrazino nicotinamide (HYNIC) labeled annexin V lipocortin in lymphatic tissue of animals treated with dexamethasone [abstract]. J Nucl Med. 1997; 38(suppl):268P.

2. Blankenberg F G, Katsikis P D, Tait J F et al. In vivo detection and imaging of phosphatidylserine expression during programmed cell death. Proc Natl Acad Sci U S A. 1998;95:6349-6354.

3. Koopman G, Reutelingsperger C P, Kuijten G A, Keehnen R M, Pals S T, van Oers M H. Annexin V for flow cytometric detection of phosphatidylserine expression on B cells undergoing apoptosis. Blood. 1994;84:1415-1420.

4. Bohn H, Kraus W. Isolation and characterization of a new placenta specific protein (PP10). Arch Gynecol. 1979;227:125-134.

5. Reutelingsperger C P, Hornstra G, Hemker H C. Isolation and partial purification of a novel anticoagulant from arteries of human umbilical cord. Eur J Biochem. 1985;151:625-629.

6. Rescher U, Zobiack N, Gerke V. Intact Ca(2+)-binding sites are required for targeting of annexin 1 to endosomal membranes in living HeLa cells. J Cell Sci. 2000;113:3931-3938.

7. Boon J M, Smith B D. Facilitated phosphatidylcholine flip-flop across erythrocyte membranes using low molecular weight synthetic translocases. J Am Chem Soc. 2001;123:6221-6226.

8. Andree H A, Stuart M C, Hermens W T, Reutelingsperger C P, Hemker H C, Frederik P M, Willems G M. J Biol Chem 1992 Sep 5;267(25):17907-12.

9. Tait J F, Gibson D, Fujikawa K. Phospholipid binding properties of human placental anticoagulant protein-I, a member of the lipocortin family. J Biol Chem. 1989;264:7944-7949.

10. Swairjo M A, Concha N O, Kaetzel M A, Dedman J R, Seaton B A. Ca(2+)-bridging mechanism and phospholipid head group recognition in the membrane-binding protein annexin V. Nat Struct Biol. 1995;2:968-974.

11. Tait J F, Gibson D F, Smith C. Measurement of the affinity and cooperativity of annexin V-membrane binding under conditions of low membrane occupancy. Anal Biochem. 2004; 329: 112-119.

12. Nicolson D W, Thornberry N A. Caspases: killer proteases. TIBS. 1997; 22: 299-306.

13. Cohen G M. Caspases: the executioners of apoptosis. J Biochem. 1997; 326: 1-16.

14. Thornberry N A, Lazebnik Y. Caspases: enemies within. Science. 1998; 282: 1312-1316.

15. Haberkorn U, Kinscherf R, Krammer P H, et al. Investigation of a potential scintigraphic marker of apoptosis: radioiodinated Z-Val-Ala-DL-Asp(o-methyl)-fluoromethylketone. Nucl Med Biol. 2001; 28: 793-798.

16. Bauer C, Bauder-Wuest U, Mier W, Haberkorn U, Eisenhut M. Investigation of ¹³¹I labelled Peptides as Caspases Substrates for Apoptosis Imaging. J Nucl Med 2005; 46: 1066-1074.

17. Kamp D, T. Sieberg and C. W. M. Haest, Biochemistry, 2001, 40, 9438.

18. Zweifach A, Biochem. J., 2000, 349, 255.

19. Dachary-Prigent J, J. M. Pasquet, J. M. Freyssinet and A. T. Nurden, Biochemistry, 1995, 34, 11625.

20. Hanshaw R G, E. J. O'Neil, M. Foley, R. T. Carpenter and B. D. Smith, J. Mater. Chem., 2005, 15, 2707.

21. Hanshaw R G, C. Lakshmi, T. N. Lambert, J. R. Johnson and B. D. Smith, ChemBioChem, 2005, 12, 2214.

22. Lakshmi C, R. G. Hanshaw, B. D. Smith, Tetrahedron 2004, 60, 11307-11315.

23. Peng W et al., Bioorg. Chem. 2005, 33, 374-385.

24. Adamczyk M, Fishpaugh J R, Heuser K J. Bioconjugate Chem. 1997, 8, 253-255.

25. Wester H J, Herz M, Weber W, Heiss P, Senekowitsch-Schmidtke R, Schwaiger M, Stocklin G. Synthesis and radiopharmacology of O—(2-[18F]fluoroethyl)-L-tyrosine for tumor imaging. J Nucl Med 1999 Jan;40(1):205-12.

26. Bauer C, Bauder-Wuest U, Mier W, Haberkorn U, Eisenhut M. Investigation of ¹³¹I labelled Peptides as Caspases Substrates for Apoptosis Imaging. J Nucl Med 2005; 46: 1066-1074.

27. Chunfu Z, Jugold M, Woenne E C, Lammers T, Morgenstern B, Mueller M M, Zentgraf H, Bock M, Eisenhut M, Semmler W, and Kiessling f. Specific Targeting of Tumor Angiogenesis by RGD-Conjugated Ultrasmall Superparamagnetic Iron Oxide Particles Using a Clinical 1.5-T Magnetic Resonance Scanner. Cancer Res 2007; 67: 1555-1562.

28. Eisenhut M., U. Haberkorn. Invited perspective: The molecular position of radiolabels and its impact on functional integrity of proteins. J Nucl Med 2006; 47: 1400-1402

29. G B Saha (Editor), Basics of PET Imaging—Physics, Chemistry, and Regulations, Springer New York 2005, Chapter 6: Cyclotron and Production of PET Radionuclides)

30. Graham Lappin, Simon Temple, Radiotracers in Drug Development, CRC Taylor & Francis 2006, Chapter 12.1.5 Choice of radionuclide for Gamma Scintigraphy and SPECT

31. Kanduc D et al, Cell death: apoptosis versus necrosis (review), Int J Oncol (2002) 21: 165-70.

1. A compound characterized in that the compound comprises
at least one cyclic polyamine unit (C) for the complexation of bi- or trivalent metal ion or complexing bi- or trivalent metal ion
and pharmaceutical salt, diastereomere and enantiomere thereof.

2. The compound according to claim 1 wherein the compound comprises further
at least one Tag (T) or linker (L)-Tag (T), and/or
at least one Bridge unit (B) or a bound,

3. The compound according to claim 1 wherein the cyclic polyamine unit (C) is a
substituted or non-substituted moiety having from 9 to 20 ring members and from 3 to 6 amine nitrogens in the ring spaced by 2 or more carbon atoms from each other.

4. The compound according to claim 3 wherein the cyclic polyamine unit (C) is selected from cyclen of formula Ia,
cyclam of formula Ib and
cyclen-cyclam mix of formula Ic 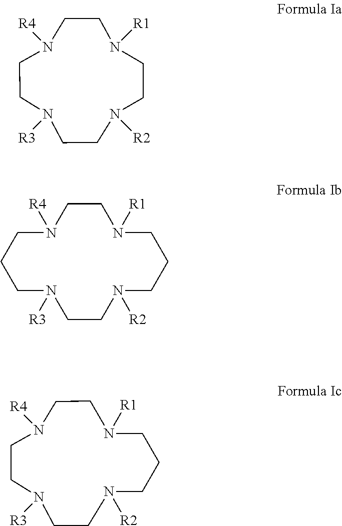
wherein R1, R2, R3 and R4 are independently from each other selected from bond, Hydrogen, C₁-C₆ alkyl, C₁-C₆ alkoxy or Alkyl—COO—, Aryl—COO—.

5. The compound according to claim 1, wherein the bivalent metal ion is chosen from the group consisting of Zn²⁺, Ca²⁺, Cu²⁺, Be²⁺, Mg²⁺, Sr²⁺, Ba²⁺, Cd²⁺, Cr²⁺, Mn²⁺, Fe²⁺, Co²⁺, Ni²⁺, Pb²⁺ and Hg²⁺.

6. The compound according to claim 1, wherein the trivalent metal ion is chosen from the group consisting of Al³⁺, Ga³⁺, In³⁺, Cr³⁺, Mn³⁺, Fe³⁺, Co³⁺and Ni³⁺.

7. The compound according to claim 1 wherein the Tag (T) or the Tag of the linker (L)-Tag (T) group is a detectable label wherein the detectable label is covalently bound to one or more cyclic polyamine units (C) or Bridge unit (B).

8. The compound according to claim 7 wherein the Tag (T) is chosen from the group comprising fluorescent dyes, enzymes, proteins, and radioactive labels.

9. The compound according to claim 8, wherein
the fluorescent dye is chosen from the group comprising of fluoresceins, rhodamines, BODIPY, Cy-3, Cy-5, and derivatives thereof.
the protein is chosen from the group comprising of peroxidases, luciferases, biotin, avidin, and antibodies or parts thereof;
the radioactive label is chosen from the group comprising of ^{99m}Tc, ¹⁸F, ¹¹C, ¹²⁴I, ¹²³I, ⁶⁴Cu²⁺, ⁶⁸Ga³⁺, ¹¹¹I³⁺, ¹⁴C, ³H, ³²P, and ³³P, preferably ¹⁸F.

10. The compound according to claim 1 wherein the Tag (T) or the linker (L)-Tag (T) is bound to the cyclic polyamine unit or to the Bridge unit (B).

11. The compound according to claim 1 wherein linker (L) is selected from
C₁-C₆ alkyl, O—C₁-C₆ alkyl, C₁-C₆ alkoxy, C₁-C₆ alkylene, (CH₂CH₂O)ₙCH₂CH₂, substituted or non-substituted aryl, substituted or non-substituted O—aryl, substituted or non-substituted heteroaryl, (CH₂)ₙPh, (CH₂CH₂CH₂O)ₙ CH₂CH₂, CO(CH₂)ₙ,[O(CH₂)ₙ—O(CH₂)ₙ]ₘ, or —O(CH₂)ₙ, [O(CH₂)ₙ—O(CH₂)ₙ]ₘ wherein Ph=phenyl and n or m=1, 2, 3, 4, 5, 6.

12. The compound according to claim 1 wherein
the Bridge unit (B) is connecting the cyclic polyamine unit (C) when there are at least two cyclic polyamine units (C) or
the Bridge unit (B) is bound to a single cyclic polyamine unit (C).

13. The compound according to claim 12 wherein the Bridge unit (B) is selected from the group comprising the substituted or non-substituted moiety described below 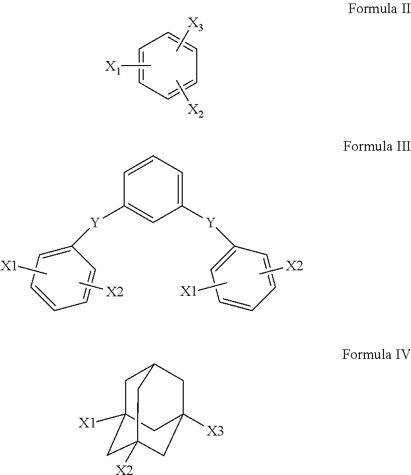
wherein X1, X2 and X3 are independently from each other a H, C₁-C₆ alkyl, C₁-C₆ alkoxy, C₁-C₆ alkylene, C(O), [O(CH₂)ₙ]ₘ, (CH₂CH₂O)ₘ, CH₂OPhCH₂, (CH₂CH₂CH₂O)ₘ, (CONHPhCH₂), (CONH(CH₂CH₂O)₂CH₂CH₂), [O(CH₂)ₙ]ₘ—NH₂, or C(O)NH—[(CH₂)ₙ—O]ₘ, Ph=phenyl, n=2 to 6 and m=1 to 6,
Formula V′ is a linear peptide consisting of α-amino acids and
Formula V′a is a linear peptide consisting of α-amino acids.

14. A pharmaceutical composition, comprising a compound of claim 1 and optionally a suitable carrier and/or additive.

15. A method comprising using a compound according to claim 1 for binding phosphatidylserine.

16. A method comprising using a compound according to claim 1 for cell death (apoptosis, necrosis) imaging.

17. The compound according to claim 1 for the manufacture of a pharmaceutical composition for cell death (apoptosis, necrosis) imaging.

18. The compound according to claim 17 wherein imaging is positron emission tomography (PET) imaging, single-photon emission computed tomography (SPECT) imaging, magnetic resonance imaging (MRI), or optical imaging.

19. A method according to claim 15, for assessing the status of a tumor, an injury, a stroke, a heart attack, in inflammation, an infection, or the response of a patient suffering from a tumor, from an infection, and/or from an inflammation, from injury, from stroke, from heart attack, to an anti-tumor therapy, to anti-infection therapy and/or to anti-inflammatory therapy, respectively.

20. A method for detecting the progression of a tumor, of an injury, of a stroke, of a heart attack, of an infection, and/or of an inflammation in a tissue of a patient, comprising
a) administering a compound according to claim 1 to a patient, and
b) measuring the amount or the concentration of the compound in the tissue.

21. A method for obtaining a compound according to claim 1.

22. A kit comprising a compound of claim 1.
